(19) United States
(12) Patent Application Publication
(10) Pub. No.: US 2014/0137290 A1
(43) Pub. Date: May 15, 2014

(54) MOLECULAR CLOCK MECHANISM OF HYBRID VIGOR

(71) Applicants: Z. Jeffrey Chen, Austin, TX (US); Eun-Deok Kim, Austin, TX (US); Zhongfu Ni, Beijing (CN)

(72) Inventors: Z. Jeffrey Chen, Austin, TX (US); Eun-Deok Kim, Austin, TX (US); Zhongfu Ni, Beijing (CN)

(21) Appl. No.: 14/147,408

(22) Filed: Jan. 3, 2014

Related U.S. Application Data
(63) Continuation of application No. 13/086,173, filed on Apr. 13, 2011.
Application No. 13/086,173 is a continuation-in-part of application No. PCT/US2009/060487, filed on Oct. 13, 2009.
(60) Provisional application No. 61/104,952, filed on Oct. 13, 2008.

(51) Int. Cl. C12N 15/82

(52) U.S. Cl. CPC C12N 15/8261
USPC 800/278

(57) ABSTRACT

Methods are provided including methods of promoting growth vigor in plants. In one embodiment, a method for promoting growth vigor in a plant comprises providing a plant comprising a circadian clock gene; and modifying expression of the circadian clock gene or modifying activity of a protein produced by the circadian clock gene so as to modify a flowering time of the plant; modify a starch, sugar, chlorophyll, metabolite, or nutrient content of the plant, or increase biomass or yield of the plant. In some embodiments, methods are provided including preparing a transgenic plant and using circadian clock genes as DNA and/or expression markers to select and predict the best combinations of parents to make hybrid plants with enhanced vigor.

### CROSS-REFERENCE TO RELATED APPLICATIONS

This application is a continuation application of co-pending U.S. patent application Ser. No. 13/086,173 filed Apr. 13, 2011; which is a continuation-in-part of International Application No. PCT/US2009/060487, filed Oct. 13, 2009, which claims the benefit of U.S. Provisional Application Ser. No. 61/104,952 filed Oct. 13, 2008, the entire disclosures of which are incorporated herein by reference.

### STATEMENT OF GOVERNMENT INTEREST

This disclosure was made with support under Grant Number GM067015, awarded by the National Institute of Health and Grant Number DBI0733857, awarded by the National Science Foundation. The U.S. government has certain rights in the invention.

### BACKGROUND

The present disclosure, according to certain embodiments, generally relates to methods of promoting growth vigor in plants. More specifically, in certain embodiments, the present disclosure provides methods for modifying circadian-rhythm gene expression in plants to modify flowering time and/or promote, inter alia, growth vigor, including higher plant content of starch, sugar, and chlorophyll, and/or increase biomass, stature, metabolites, and/or yield.

Scientists have known for years—since Charles Darwin made the discovery in 1876—that hybrid plants or animals grow stronger and larger than their parents. This is also true for polyploids, or plants that have two or more sets of chromosomes. This phenomenon is generally known as hybrid vigor or heterosis.

Hybrids and polyploids (whole genome duplication) are common in plants and animals. Some crops, such as corn and rice, are grown mainly as hybrids, and many others such as wheat, cotton, and oilseed rape are grown as polyploids. Hybrids are formed by hybridizing different strains, varieties, or species. Polyploids are formed by duplicating a genome within the same species (known as autopolyploids, such as potato, alfalfa, and sugarcane) or between different species (known as allopolyploids, such as wheat, cotton, and oilseed rape). The common occurrence of hybrids and polyploids suggests an evolutionary advantage of having additional genetic material for natural selection and plant domestication, which may lead to increased growth vigor and adaptation in many hybrid and polyploid plants, vegetables, and crops. The molecular basis for this advantage was previously unknown.

In plants and animals, it is believed that circadian clock regulators mediate physiological and metabolic processes that are associated with growth and fitness. These regulators provide positive and negative feedback regulation for maintaining proper internal clocks, which in turn controls the expression of downstream genes in various physiological and metabolic pathways. In plants, circadian clock regulators and their regulatory networks are conserved.

Growth vigor and biomass in plants are affected by rates of photosynthesis, carbon fixation, and starch metabolism. An increase in the synthesis of chlorophylls generally correlates to a higher content of starch and sugar, as well as increased growth, biomass, and yield. Many genes responsible for light-signaling pathways, flowering time, chlorophyll biosynthesis, carbon fixation, and starch metabolism are known or predicted to be controlled by circadian clock regulators. However, how the circadian clock regulators affect growth vigor in hybrids and polyploid plants is unknown.

### SUMMARY

The present disclosure, according to certain embodiments, generally relates to methods of promoting growth vigor in plants. More specifically, in certain embodiments, the present disclosure provides methods for modifying circadian-rhythm gene expression in plants to modify flowering time and/or promote, inter alia, growth vigor, including higher plant content of starch, sugar, and chlorophyll, and/or increase biomass, stature, metabolites, and/or yield.

The present disclosure, according to certain embodiments, discovers a link between circadian clock regulators and growth vigor. Certain circadian clock genes (“CCGs”), such as CIRCADIAN CLOCK ASSOCIATED 1 (“CCA1”), LATE ELONGATED HYPOCOTYL (“LHY”), TIMING OF CAB EXPRESSION 1 (“TOC1”), CCA1 Hiking Expedition (CHE), and GIGANTEA (“GI”), mediate expression changes in many downstream genes and metabolic pathways associated with growth vigor. The methods of the present invention provide for modification of a CCG, or product thereof, so as to promote growth vigor, modify flowering time, and/or increase carbon fixation, biomass, stature, metabolites, and/or yield in plants.

In some embodiments, the methods of the present invention may comprise providing a plant comprising a circadian clock gene; and modifying expression of the circadian clock gene or modifying activity of a protein produced by the circadian clock gene so as to modify a flowering time of the plant; modify a starch, sugar, chlorophyll, metabolite or nutrient content of the plant, or increase biomass of the plant.

In another embodiment, the methods of the present invention may comprise comprising inhibiting CCA1 or LHY activity in a plant cell.

In yet another embodiment, the methods of the present invention may comprise enhancing TOC1, CHE or GI activity in a plant cell.

In yet another embodiment, the methods of the present invention may comprise a method of preparing a transgenic plant comprising: transforming a plant cell with one or more circadian clock genes so as to create a transformed plant cell; and generating a plant from the transformed plant cell.

In yet another embodiment, the methods of the present invention may comprise a method of preparing a transgenic plant comprising: transforming a plant cell with one or more genes regulated by a circadian clock gene so as to create a transformed plant cell; and generating a plant from the transformed plant cell.

In yet another embodiment, the methods of the present invention may comprise a method of using circadian clock genes as DNA and/or gene expression markers to select and predict best combinations of parental lines to make hybrids that increase growth vigor.

The features and advantages of the present invention will be readily apparent to those skilled in the art upon a reading of the description of the embodiments that follows.

### DRAWINGS

A more complete understanding of this disclosure may be acquired by referring to the following description taken in combination with the accompanying figures.

FIG. 1a is a graph representing the qRT-PCR analysis of CCA1 expression in a 24-hour period, according to specific example embodiments of the present disclosure.

FIG. 1b is a graph representing the qRT-PCR analysis of TOC1 expression in a 24-hour period, according to specific example embodiments of the present disclosure.

FIG. 1c is an image of a gel depicting the repression of A. thaliana CCA1 and LHY and upregulation of A. thaliana TOC1 and GI in the allotetraploids, according to specific example embodiments of the present disclosure.

FIG. 1d is an image of the chromatin immunoprecipitation (ChIP) analysis results of CCA1, LHY, TOC1 and GI, according to specific example embodiments of the present disclosure.

FIG. 2a is a graph showing increase of chlorophyll content in allotetraploids, according to specific example embodiments of the present disclosure.

FIG. 2b is a schematic diagram of the starch metabolic pathways in the chloroplast (circled) and cytoplasm, according to specific example embodiments of the present disclosure.

FIG. 2c is a gel image depicting the upregulation of PORA and PORB in the allotetraploids at ZT6 by Reverse Transcriptase (RT)-PCR, according to the specific example embodiments of the present disclosure.

FIG. 2d is a gel image depicting the upregulation of starch metabolic genes in allotetraploids at ZT6, according to the specific example embodiments of the present disclosure.

FIG. 3a is an image showing starch staining in A. thaliana (At4), A. arenosa (Aa), and allotetraploid (Allo733) at ZT0, ZT6, and ZT15, according to specific example embodiments of the present disclosure.

FIG. 3b is a graph summarizing the increased starch content in allotetraploids at ZT6, according to specific example embodiments of the present disclosure.

FIG. 3c is a graph summarizing the increased sugar content in allotetraploids at ZT6, according to specific example embodiments of the present disclosure.

FIG. 3d is a picture depicting morphological vigor in F₁ hybrids between A. thaliana Columbia (Col) and C24, according to specific example embodiments of the present disclosure.

FIG. 3e is a graph summarizing the increased chlorophyll (ZT6, left) and starch (ZT15, right) accumulation in F₁, according to specific example embodiments of the present disclosure.

FIG. 3f is a graph showing CCA1 protein levels changed in allotetraploids (Allo733 and Allo738) and their progenitors (At4 and Aa), and A. thaliana transgenics overexpressing CCA1 at ZT6 and ZT0, according to specific example embodiments of the present disclosure.

FIG. 3g is a gel image showing the specific CCA1 binding activity to EE of downstream genes (TOC1 and PORB) in vitro, according to specific example embodiments of the present disclosure.

FIG. 3h is an image of the ChIP analysis results of endogenous CCA1 binding to the TOC1 promoter, according to specific example embodiments of the present disclosure.

FIG. 4a contains graphs representing the relative expression levels (R.E.L.) of CCA1, reduced chlorophyll and starch accumulation in TOC1:CCA1 lines, according to specific example embodiments of the present disclosure.

FIG. 4b contains graphs representing the reduced CCA1 expression and increased starch content in cca1-11 and cca1-11 lhy-21 mutants, according to specific example embodiments of the present disclosure.

FIG. 4c is a graph and a gel image showing the decreased expression of CCA1 mRNA and protein in TOC1:cca1-RNAi transgenic plants, according to specific example embodiments of the present disclosure.

FIG. 4d is a graph depicting the increased starch content in TOC1:cca1-RNAi lines, according to specific example embodiments of the present disclosure.

FIG. 4e is a schematic diagram of a model for growth vigor and increased biomass. Chromatin-mediated changes in internal clock regulators in hybrids or allotetraploids lead to up- and down-regulation and downstream genes and output traits at noon (sun) and dusk (moon), according to specific example embodiments of the present disclosure.

FIG. 5 is an image depicting morphological vigor of Arabidopsis allotetraploids, according to specific example embodiments of the present disclosure.

FIG. 6a contains a graph showing the expression of circadian clock regulators (LHY) in a 24-hour period using zeitgeber time starting from dawn, according to specific example embodiments of the present disclosure.

FIG. 6b contains a graph showing the expression of circadian clock regulators (GI) in a 24-hour period using zeitgeber time starting from dawn, according to specific example embodiments of the present disclosure.

FIG. 6c is a gel image showing the expression of circadian clock regulators (LHY and GI) in a 24-hour period using zeitgeber time starting from dawn, according to specific example embodiments of the present disclosure.

FIG. 6d contains a graph representing the relative expression levels (R.E.L.) of CCA1, LHY and GI, according to specific example embodiments of the present disclosure.

FIG. 7a contains a graph representing expression of a circadian clock regulator (CCA1) in Arabidopsis thaliana hybrids and their parents, according to specific example embodiments of the present disclosure.

FIG. 7b contains a graph representing expression of a circadian clock regulator (LHY) in Arabidopsis thaliana hybrids and their parents, according to specific example embodiments of the present disclosure.

FIG. 7c contains a graph representing expression of a circadian clock regulator (TOC1) in Arabidopsis thaliana hybrids and their parents, according to specific example embodiments of the present disclosure.

FIG. 8 is an image showing the results of the electrophoretic mobility shift assay (EMSA) showing competitive binding of recombinant CCA1 to DPE1, GWD3, and PORA promoter fragments, according to specific example embodiments of the present disclosure.

FIG. 9a characterizes CCA1 overexpression lines driven by 35S and TOC1 promoters showing reduced chlorophyll and starch content in CCA1-OX and TOC1:CCA1 transgenic plants, according to specific example embodiments of the present disclosure.

FIG. 9b depicts a ProTOC1:CCA1 construct, according to specific example embodiments of the present disclosure.

FIG. 9c is a graph depicting the reduced chlorophyll content in the CCA1-OX line and TOC1:CCA1 transgenic plants at ZT9 (left) and decreased starch content in the leaves of TOC1:CCA1 transgenic lines at ZT6 (right).

FIG. 10a contains a graph representing the relative expression levels of downstream genes in TOC1:CCA1 transgenic plants, according to specific example embodiments of the present disclosure.

FIG. 10b contains a graph representing the relative expression levels of CCA1 and downstream genes in cca1, and cca1 lhy mutants, according to specific example embodiments of the present disclosure.

FIG. 10c depicts a ProTOC1:cca1-RNAi construct, according to specific example embodiments of the present disclosure.

FIG. 10d is a picture depicting some TOC1:cca1-RNAi transgenic plants, according to specific example embodiments of the present disclosure.

FIG. 10e contains a graph representing the relative expression levels of downstream genes in TOC1:cca1-RNAi transgenic plants, according to specific example embodiments of the present disclosure.

FIG. 11 contains photos and diagrams depicting heterosis in maize seedlings and conservation of circadian clock regulators in plants (Arabidopsis, maize, rice, sorghum, grape, and poplar), according to specific example embodiments of the present disclosure.

FIG. 11a is an image depicting growth vigor in maize F₁ seedlings from a cross between Mo17 and B73. Two reciprocal F₁ hybrids are shown in the middle. By convention, the maternal parent appears first in a genetic cross.

FIG. 11b is an image showing growth vigor in maize F₁ seedlings from reciprocal crosses between B73 and W22.

FIG. 11c is a diagram depicting the phylogenetic tree of AtLHY, AtCCA1, ZmLHY1, ZmLHY2, SbMYB1, OsLHY, VvCCA1/LHY, and PnLHY that are highly conserved among these plants. At: Arabidopsis thaliana; Zm: Zea mays (maize); Sb: Sorghum bicolor (sorghum); Os: Oryza sativa (rice); Vv: Vitis vinifera (grapevine); and Pn: Populus trichocarpa (poplar).

FIG. 11d is a diagram depicting the phylogenetic tree of TOC1 and related PRR genes, AtTOC1, OsTOC1, ZmTOC1, APRR3, APRR5, APRR7, APRR9, OsPRR37, OsPRR59, OsPRR73, OsPRR95, ZmPRR73, and ZmPRR95 that are highly conserved among these plants. APRR: Arabidopsis clock-associated pseudo-response regulators.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as defined by the appended claims.

### DESCRIPTION

The present disclosure, according to certain embodiments, generally relates to methods of promoting growth vigor in plants. More specifically, in certain embodiments, the present disclosure provides methods for modifying circadian-rhythm gene expression in plants to modify flowering time and/or promote, inter alia, growth vigor, including higher plant content of starch, sugar, and chlorophyll, and/or increase biomass, stature, metabolites, and/or yield.

According to some embodiments, the present disclosure includes repression of certain negative circadian clock regulators and/or upregulation of certain positive circadian clock regulators in plants, including hybrids and/or polyploids, to promote the expression of downstream genes whose products may be involved in many biological processes including, but not limited to, light-signaling, chlorophyll biosynthesis, starch and sugar metabolism, and flowering-time. In some embodiments, this repression and/or upregulation may occur during the day. As a result, the plants may accumulate more chlorophyll, starch, sugar and other carbohydrates, and more metabolites, grow larger and healthier, and produce more fruits and seeds. In general, modifying the expression of circadian clock genes changes the growth vigor in plants.

Circadian clocks may allow organisms to adapt to many different types of environmental changes and also may provide a mechanism to mediate metabolic pathways and generally increase fitness of an organism. In plants, circadian clock performance may be attributed to the products of certain circadian clock genes (“CCGs”), such as CIRCADIAN CLOCK ASSOCIATED 1 (“CCA1”), LATE ELONGATED HYPOCOTYL (“LHY”), TIMING OF CAB EXPRESSION 1 (“TOC1”), CCA1 Hiking Expedition (“CHE”), GIGANTEA (“GI”) and other related genes, which are now believed to be at least partially responsible for mediating expression changes in many downstream genes and pathways associated with growth vigor. As used herein, the term “circadian clock gene” refers to CCA1, LHY, TOC1, CHE, GI and any related gene or any gene that functions in the same manner as CCA1, LHY, TOC1, CHE or GI. In one embodiment, the present disclosure provides methods for modification of one or more circadian clock genes, such as CCA1,LHY, TOC1, CHE, and GI, and/or the products of the genes, in an effort to improve growth vigor, to modify flowering time, and/or to create increased biomass in plants. In another embodiment, CCA1, LHY, TOC1, CHE, GI and other circadian clock genes may be used as molecular markers to predict growth vigor in hybrids and polyploids of crops, vegetables, fruits, energy crops, and trees. In some embodiments, a plant may be modified in accordance with the methods of the present invention so as to have desirable characteristics such as, a higher starch content, sugar content, chlorophyll content, metabolite content, and/or nutrient content, as compared to non-modified plants. Furthermore, the methods of the present invention may allow for improved plant robustness, biomass, stature, yield and quality of crops.

Generally speaking, CCA1, LHY, TOC1, CHE, and GI production may be regulated through a circular feedback pathway that maintains the rhythm, amplitude, and/or period of an organism's circadian clock. CCA1 and LHY are MYB-domain transcription factors with partially redundant functions that are expressed at relatively low levels during the day and relatively high levels at night. Contrastingly, TOC1 CHE, and GI are expressed at relatively high levels during the day but low levels at night. The circular feedback pathway involving these proteins is such that CCA1 and LHY negatively regulate TOC1 and GI expression, whereas TOC1 binds to the CCA1 promoter and interacts with CHE, positively regulating CCA1 and LHY expression. That is, TOC1, CHE, and GI are the reciprocal regulators for CCA1 and LHY, and therefore enhanced TOC1, CHE, and GI activity parallels decreased CCA1 and LHY activity. While not being bound to any particular theory, it is believed that CCA1 and LHY may bind to a CCA1 binding site (CBS) or evening element (EE) present on a particular downstream gene which may be responsible for, inter alia, photosynthesis, sugar metabolism, starch production, and chlorophyll production.

As a result of this circular feedback pathway, it has been discovered that the down-regulation of CCA1 and/or LHY promotes growth vigor, while their up-regulation reduces growth vigor. Likewise, it has been discovered that the up-regulation of TOC1, CHE, and/or GI promotes growth vigor, while their down-regulation reduces growth vigor. This is most likely a result of their mediating expression changes in downstream genes and pathways. Furthermore, overexpressing CCA1 is generally related to late flowering, whereas down-regulating CCA1 is related to early flowering. Changes in flowering time affect vegetative growth and plant biomass.

In some embodiments, the methods of the present invention comprise inhibiting CCA1 and/or LHY activity in one or more plant cells. In one embodiment, CCA1 or LHY activity may be inhibited by administering a CCA1 or LHY inhibitor. Suitable CCA1 or LHY inhibitors for use in the methods of the present invention may be any inhibitor of CCA1 or LHY. As used herein, the term “CCA1 or LHY inhibitor” refers to a compound capable of at least temporarily reducing the activity of CCA1 or LHY. In some embodiments, suitable CCA1 or LHY inhibitors may be capable of inhibiting CCA1 or LHY activity by blocking the catalytic domain of CCA1 or LHY. Examples of such inhibitors may include, but are not limited to anti-CCA1 or LHY antibodies, Actinomycin D, Alpha Amanitin, and Cordycepin.

In some embodiments, the methods of present invention comprise inhibiting the activity of CCA1 and/or LHY by moving the CCA1 gene, LHY gene or its products from one plant species to another. For example, CCA1 or LHY can be cloned from one plant species and transformed into another plant using transgenic approaches. Alternatively, CCA1 or LHY from one species can be introgressed into a related species using breeding schemes such as wide hybridization and backcrossing. In other embodiments, the methods of present invention comprise inhibiting the activity of CCA1 and/or LHY by hybridizing two plants within the same species or between two different plant species or genera. Hybrids refer to offspring formed within the same species; intraspecific hybrids refer to the offspring formed between the sub-species; and interspecific or intergeneric hybrids refer to offspring formed between species or between genera. Hybridizing different plant strains, species, and/or genera with different genetic alleles or loci of circadian clock genes may generate a genetic condition of heterozygotes that induce altered expression patterns of circadian clock genes such as CCA1 and LHY. One common practice is to cross-hybridize a plant with a closely related plant species and breed offspring for the intrgression of one or more circadian clock genes from the related species into a plant or crop for cultivation. The CCA1 and/or LHY can also change in polyploid plants in which the number of chromosomes of the plant is increased or decreased.

In some embodiments, the methods of present invention comprise inhibiting the activity of CCA1 and/or LHY by applying chemicals and/or enzymes that modify CCA1 or LHY in one or more plant cells. In some embodiments, a chemical may be provided that degrades CCA1 or LHY. In some embodiments, a chemical may be provided that decreases the half-life of CCA1 or LHY. In some embodiments, a chemical may be provided that inhibits CCA1 or LHY function. Examples of chemicals suitable for use in the methods of the present invention may include a chromatin reagent, such as 5′-aza-2′-deoxycytidine (aza-dC) and its derivatives, trichostatin A (TSA), CHAHA, HC-toxin, and/or sodium butyrate.

In some embodiments, the methods of present invention comprise inhibiting the activity of CCA1 and/or LHY by overexpressing or down-regulating the expression of proteins, elements, and factors that interact with CCA1 and/or LHY such as, for example, TOC1, CHE, GI, ELF4, ELF3, LUX, PHY, TIC. The methods include but are not limited to the use of mutagens, genetic manipulations, homologous recombination, RNA interference (RNAi) that knock-out, silence, or repress CCA1 or LHY activity or the use of transgenes to over-express positive regulators such as TOC1, CHE, GI, or downstream genes in light-signaling, chlorophyll, and starch metabolism.

In some embodiments, the methods of the present invention comprise inhibiting the activity of CCA1 and/or LHY by blocking gene expression of CCA1 and/or LHY. Gene expression is the process by which a nucleic acid sequence of a gene is converted into a functional gene product, such as protein or RNA. Blocking expression, transcription or translation of CCA1 or LHY are additional mechanisms of inhibition. Several steps in the gene expression process may be modulated to produce CCA1 or LHY inhibition. For example, in some embodiments, an inhibitor to block CCA1 or LHY transcription, the process by which the nucleic acid sequence is converted to RNA, may be administered. Examples of these transcription inhibitors include but are not limited to Actinomycin D, Alpha Amanitin, and Cordycepin. Similarly, an inhibitor of CCA1 or LHY translation, the process by which messenger RNA is translated into a specific polypeptide, may be administered. Examples of translation inhibitors include but are not limited to Cycloheximide, Cordycepin, Puromycin dihydrochloride, and Hygromycin B.

In some embodiments, the methods of the present invention comprise enhancing the activity of TOC1, CHE, and/or GI in one or more plant cells by administering a TOC1, CHE or GI enhancer. TOC1 and CHE are reciprocal regulators for CCA1, and therefore enhanced TOC1 or CHE activity parallels decreased CCA1 activity. Suitable TOC1, CHE, or GI enhancers for use in the methods of the present invention may be any enhancer of TOC1, CHE or GI. As used herein, the term “TOC1, CHE, or GI enhancer” refers to a compound capable of at least temporarily enhancing the activity of TOC1, CHE, or GI. In some embodiments, suitable TOC1, CHE, or GI enhancers may be capable of enhancing TOC1, CHE, or G1 activity by decreasing expression of their negative regulators such as CCA1 or LHY or by increasing the number of promoter elements such as CBS and evening elements.

In some embodiments, the methods of present invention comprise enhancing the activity of TOC1, CHE and/or GI by moving the TOC1 gene, CHE gene, GI gene or its products from one plant species to another. For example, TOC1, CHE, or GI may be cloned from one plant species and transformed into another plant using transgenic approaches. Alternatively, TOC1, CHE, or GI from one species can be introgressed into a related species using breeding schemes such as wide hybridization and backcrossing. In other embodiments, the methods of present invention may comprise enhancing the activity of TOC1, CHE, and/or GI by hybridizing two plants within the same species or between two different plant species or genera. Hybrids refer to offspring formed within the same species; intraspecific hybrids refer to the offspring formed between the sub-species; and interspecific or intergeneric hybrids refer to offspring formed between species or between genera. Hybridizing different plant strains and/or species that contain different genetic alleles or loci of circadian clock genes generates a genetic condition of heterozygotes that induce altered expression patterns of circadian clock genes such as TOC1, CHE, and/or GI. The clock regulators can also change in polyploid plants in which the number of chromosomes of the plants is increased or decreased.

In some embodiments, the methods of present invention comprise enhancing the activity of TOC1, CHE and/or GI by applying chemicals and/or enzymes that modify the expression of TOC1, CHE and/or GI in one or more plant cells. In some embodiments, a chemical or method may be provided that decreases the rate of degradation of TOC1, CHE or GI. In some embodiments, a chemical or method may be provided that increases the half-life of TOC1, CHE or GI. In some embodiments, a chemical or method may be provided that enhances TOC1, CHE or GI function. Examples of chemicals suitable for use in the methods of the present invention may include those that cause overexpression of TOC1, CHE, GI using transgenic approaches.

In some embodiments, the methods of the present invention comprise enhancing the activity of TOC1, CHE and/or G1 by increasing expression of TOC1, CHE and/or GI. As previously mentioned, gene expression is the process by which a nucleic acid sequence of a gene is converted into a functional gene product, such as protein or RNA. Enhancing expression, transcription or translation of TOC1, CHE and/or GI are additional mechanisms of enhancement. Several steps in the gene expression process may be modulated to produce TOC1 or GI enhancement. For example, in some embodiments, an enhancer to increase TOC1, CHE and/or GI transcription may be administered. Similarly, an enhancer of TOC1, CHE, or GI translation may be administered. These agents include but are not limited to chromatin reagents such as such as 5′-aza-2′-deoxycytidine (aza-dC) and its derivatives, trichostatin A (TSA), CHAHA, HC-toxin, and/or sodium butyrate.

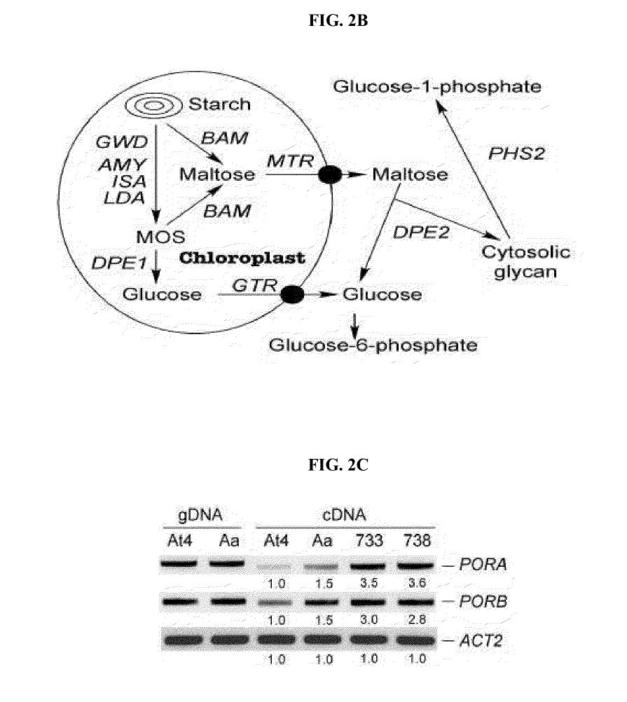

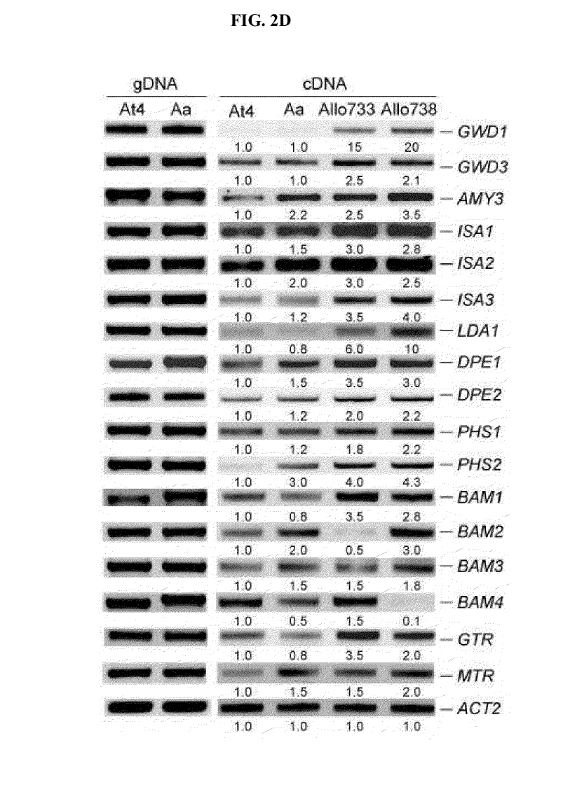

The present disclosure provides, according to one embodiment, methods comprising using CCA1 and/or LHY, or similar circadian clock regulators, in plants to modify expression of downstream genes that possess EE or CBS motifs. Examples of downstream genes that possess EE or CBS motifs include the genes that are responsible for photosynthesis, starch and sugar metabolism, flowering time, other carbohydrates and secondary metabolites, some of which are listed in FIGS. 2b, 2d and 2d and Tables 6 and 7.

In another embodiment, the present disclosure provides a method of preparing a transgenic plant that comprises transforming a plant cell with one or more circadian clock genes so as to create a transformed plant cell and subsequently generating a plant from the transformed plant cell. For example, a circadian clock gene may be cloned from one plant species and transformed into another plant using transgenic approaches. Alternatively, a circadian clock gene from one species can be introgressed into a related species using breeding schemes such as wide hybridization and backcrossing. In other embodiments, a hybrid plant may be hybridizing two plants within the same species or between two different plant species or genera. As previously mentioned, hybrids refer to offspring formed within the same species; intraspecific hybrids refer to the offspring formed between the sub-species; and interspecific or intergeneric hybrids refer to offspring formed between species or between genera. Hybridizing different plant strains, species, and/or genera with different genetic alleles or loci of circadian clock genes may generate a genetic condition of heterozygotes that induce altered expression patterns of circadian clock genes. One common practice is to cross-hybridize a plant with a closely related plant species and breed offspring for the intrgression of one or more circadian clock genes from the related species into a plant or crop for cultivation. In some embodiments, the resulting plant may be a hybrid or a polyploid.

In another embodiment, the present disclosure provides a method of preparing a transgenic plant that comprises transforming a plant cell with one or more genes regulated by a circadian clock gene so as to create a transformed plant cell and subsequently generating a plant from the transformed plant cell. In some embodiments, circadian clock regulated genes may participate in light-signaling, hormone signaling, flowering time, or biosynthesis and metabolism of chlorophylls, starch, sugars, other carbohydrates, or a secondary metabolite, including but not limited to ELF4, ELF3, LUX, PHY, TIC, FT, FLC, PORA, PORB, AMY3, BAM1, 2 and 3, DPE1 and 2, GTR, GWD1 and 3, ISA1, 2 and 3, LDA, MEX1, and PHS1 and 2. In some embodiments, the resulting plant may be a hybrid or a polyploid.

In another embodiment, CCA1, LHY, TOC1, CHE, GI and other circadian clock genes may be used as molecular markers to predict growth vigor in hybrids and polyploids of crops, vegetables, fruits, energy crops, and trees. The degree of expression changes in certain circadian clock genes may be directly correlated with the degree of chlorophyll, starch, sugar content. In principle, any genes that are related to expression differences between a hybrid or polyploid plant and the parents can be used as genetic markers to predict the growth performance (e.g., chlorophylls, starch, sugars, metabolites, and flowering time).

Examples of plant cells suitable for use in the methods of the present invention include any plant cell having a CCG. For example, the plant cell may be a plant cell from crop plants (e.g., corn, wheat, rice, sugarcane, sorghum, millet, rye, cotton, soybean, tobacco, oilseed rape, spinach, grapes, sunflower, peanut, alfalfa, and mustard), vegetable, fruit, and energy plants (e.g., pepper, tomato, cucumber, squash, watermelon, potato, cabbage, rose, petunia, strawberry, peach, apple, orange, banana, tea, coca, cassaya, switchgrass, elephant grass, Sudan grass, Chinese tallow, clover, Jatropha curcas, and algae), trees (e.g., tea, bamboo, poplar, kiwi, willow, palm, and pine), and others such medicinal plants and herbs that grow for the harvest of plant biomass, metabolites, and nutrients. The plant cell used may be a cell in culture, or may be a cell or part of tissue or organ that is still in a plant or seed of a plant.

### EXAMPLES

### Methods

Arabidopsis allotetraploids were resynthesized by hybridizing A. thaliana with A. arenosa tetraploids, and hybrids were made by crossing C24 with Columbia. Maize hybrids were made by crossing Mo17 and B73 and by crossing B73 and W22. Unless noted otherwise, 8-15 plants (grown under 22° C. and 16-hour light/day) from each of 2-3 biological replications were pooled for the analysis of DNA, RNA, protein, chlorophyll, starch, and sugar. TOC1:CCA1 and TOC1:cca1-RNAi transgenic plants were produced using pEarlygate303 (CD694) and pCAMBIA (CD3-447) derivatives, respectively. cca1-11 (CS9378) and cca1-11 lhy-21 (CS9380) mutants were obtained from Arabidopsis Biological Resource Center (ABRC). Protein blot, EMSA, and ChIP assays were performed according to published protocols.

Plant Growth

Plant materials included A. thaliana autotetraploid (At4, ABRC accession no. CS3900), A. arenosa (Aa, CS3901), and two independently resynthesized allotetraploid lineages (Allo733 and Allo738) (CS3895-96) (F₇ to F₈). Plants for 24-hour rhythm analysis were grown for 4 weeks in 16/8-hr (light/dark) cycles and harvested at indicated zeitgeber time (ZT0=dawn). For each genotype, mature leaves from five plants were harvested every 3 hours for a period of 48 hours and frozen in liquid nitrogen. Leaves were collected prior to bolting (6-8 rosette leaves in A. thaliana, 10-12 leaves in A. arenosa, and 12-15 leaves in allotetraploids) to minimize developmental variation among genotypes. Unless noted otherwise, analyses for gene expression, chlorophyll, starch, and sugars were performed at ZT6 (noon), 6, 9, and 15.

Maize plants (inbred lines and hybrids) were grown in a growth chamber with 26° C. during the day and 20° C. at night with a light cycle of 16 hours. Leaves were harvested from a pool of 5-10 seedlings 14 days after seed germination for gene expression and biochemical assays.

CCA1 Transgenic Plants

The constitutive CCA1-overexpression line (CCA1-OX) was provided by Elaine Tobin at University of California, Los Angeles. Cloning was performed according to the protocol available at the worldwide web natureprotocols.com/2009/01/08/cloning_circadian_promoters.php, which is hereinafter described. A TOC1 (At5g61380.1) promoter fragment was amplified using A. thaliana Columbia genomic DNA and the primer pair 5′-GGGAATTCCGTGTCTTACGGTGGATGAAGTTGA-3′ (EcORI) (SEQ ID NO 1) and 5′-GGGGATCCGTTTT GTCAATCAATGGTCAAATTATGAGACGCG-3′ (BamHI) (SEQ ID NO 2) and a full-length CCA1 cDNA fragment using the primer pair: 5′-GCGGCCGGATCCATGGAGACAAATTCGTCTGGAG-3′ (BamHI) (SEQ ID NO 3) and 5′-GGCCGCTCTAGATCATGTGGAAGCTTGAGTTTC-3′ (XbaI) (SEQ ID NO 4). The TOC1 promoter fragment was fused to CCA1 cDNA and cloned into pBlueScript. The inserts were validated by sequencing and subcloned into pEarlyGate303 (CD694) using the primer pair 5′-GGGGACAAGTTTGTACAAAAAAGCAGGCTTACGTGTCTTACGGTGGATGAAGTT GA-3′ (SEQ ID NO 4) and 5′-GGGGACCACTTTGTACAAGAAAGCTGGGTCTGTGGAAGCTTGAGTTTCCAACCG-3′ (SEQ ID NO 6). The construct (ProTOC1:CCA1) was transformed into A. thaliana (Columbia) plants (FIG. 8b). One-week old T1 seedlings (two true leaves) were sprayed with basta solution (˜100 mg/L), and the positive plants were genotyped (FIG. 8). T2 transgenic plants (TOC1:CCA1) were subjected to chlorophyll, starch, and gene expression analysis.

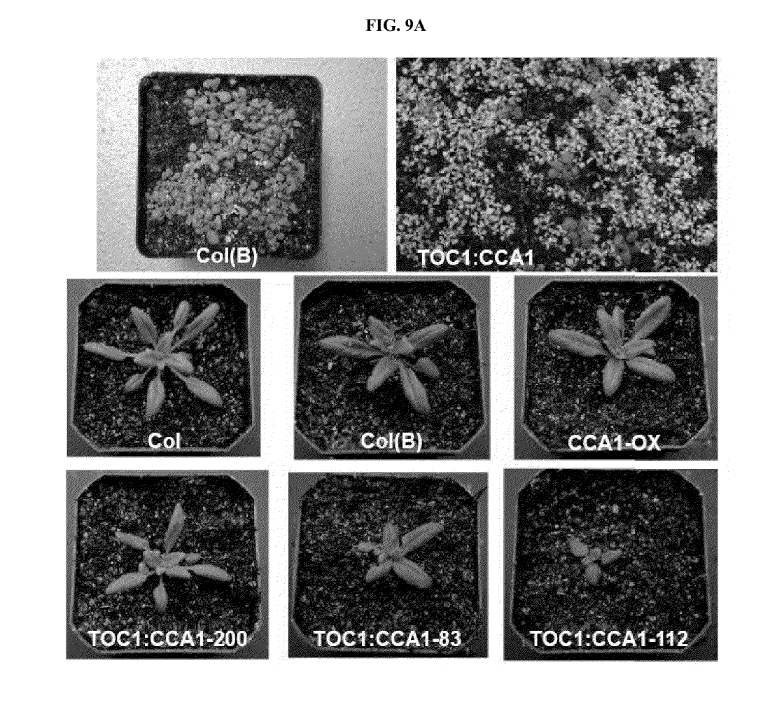

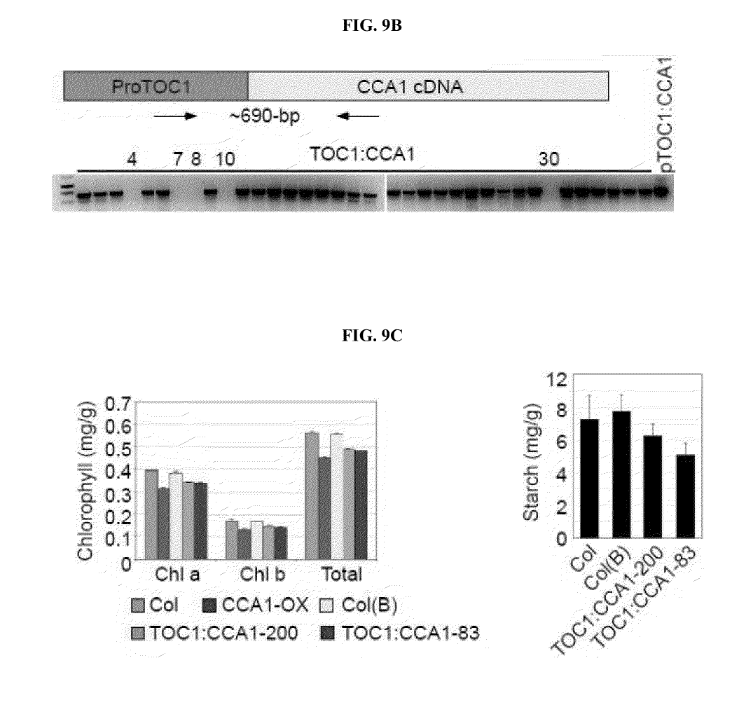

To make the TOC1:cca1-RNAi construct, a TOC1 promoter fragment (ProTOC1) was amplified using the primer pair: F-EcoRI-ProTOC1 5′-GGGAATTCCGTG TCTTACGGTGGATGAAGTTGA-3′ (SEQ ID NO 7) and R-ProTOC1-NcoI 5′-GCGGCCCCATGGGTTTT GTCAATCAATGGTCAAATTATGAGACGCG-3′ (SEQ ID NO 8) and replaced 35S promoter with ProTOC1 in pFGC5941 (CD3-447) (FIG. 9c). A 250-bp CCA1 fragment was amplified using the primer pair: F-RNAi CCA1 XbaI AscI 5′-GCGGCCTCTAGAGGCGCGCCT CTGGAAAACGGTAATGAGCAAGGA-3′ (SEQ ID NO 9) and R-RNAi CCA1 BamHI SwaI 5′-GGCCGCCCTAGGTAAATTTACACCACTAGAATCGGGAGGCCAAA-3′ (SEQ ID NO 10). The BamHI-XbaI fragment and then the AscI-SwaI fragment were subcloned into the same vector, generating two CCA1 fragments in opposite orientations (pTOC1:cca1-RNAi) (FIG. 9c). Four TOC1:cca1-RNAi T1 transgenic plants were used to analyze gene expression and starch content.

Mutant seeds of cca1-11 (CS9378) and cca1-11 lhy-21 (CS9380) were obtained from ABRC. Gene expression, chlorophyll and starch assays were performed when the mutant plants were about 3-4 weeks old and had 6-8 true leaves under 16/8 hours of day/night before bolting.

DNA and RNA Analysis

Genomic DNA was extracted using a modified protocol. Total RNA was extracted using RNeasy plantmini kits (Qiagen, Valencia, Calif.). The first-strand cDNA synthesis was performed using reverse transcriptase (RT) Superscript II (Invitrogen, Carlsbad, Calif.). An aliquot ( 1/100) of cDNA was used for quantitative RT-PCR (qRT-PCR) analysis using the primer pairs for LHY, CCA1, TOC1, and GI (Table 1) in an ABI7500 machine (Applied Biosystems, Foster City, Calif.) as previously described, except that ACT2 was used as a control to estimate the relative expression levels in three biological replications.

To distinguish locus-specific expression patterns, the RT-PCR products were amplified using the primer pairs (Table 3) and subjected to cleaved amplified polymorphism sequence (CAPS) analysis.

Semi-quantitative RT-PCR was used to determine the expression levels of the genes in chlorophyll a and b biosynthesis and starch metabolism.

Chlorophyll, Starch and Sugar Contents

The protocol for this procedure is available at the worldwide web natureprotocols.com/2009/01/08/chlorophyll_and_starch_assays_{—}1.php, which is hereinafter described. Chlorophyll was extracted in the dark with 5 ml of acetone (80%) at 4° C. from 300 mg 4-week-old seedlings. The chlorophyll content was calculated using spectrophotometric measurements at light wavelengths of 603, 645 and 663 nm and 80% acetone as a control and shown as milligram of chlorophyll per gram of fresh leaves.

Ca (mg/g)=12.7×OD663−2.69×OD645 (Chlorophyll a)

Cb (mg/g)=22.9×OD645−4.86×OD663 (Chlorophyll b)

Ca+b (mg/g)=8.02×OD663+20.20×OD645 (Chlorophyll a+b)

Starch content was measured from leaves of five plants (about 600 mg fresh weight). The leaves were boiled in 25 mL 80% (v/v) ethanol. The decolored leaves were stained in an iodine solution or ground with a mortar and pestle in 80% ethanol. Total starch in each sample was quantified using 30 μl of the insoluble carbohydrate fraction using a kit from Boehringer Mannheim (R-Biopharm, Darmstadt, Germany).

To quantify soluble sugars, 600 mg fresh leaves were extracted with 80% ethanol. The sugar concentration was determined enzymatically using Maltose/Sucrose/D-Glucose and D-Glucose/D-Fructose kits, respectively (Boehringer Mannheim, R-Biopharm) and shown as milligram of sugar per gram of fresh leaves.

Promoter Motif Analysis

DNA sequences from ˜1,500-bp upstream of the transcription start sites of the upregulated genes identified in the allotetraploids were extracted and searched for evening element (EE, AAAATATCT) (SEQ ID NO 11) or CCA1 binding site (CBS, AAAAATCT) (SEQ ID NO 12). The same method was used to analyze motifs in all genes in Arabidopsis genome. The list of 128 upregulated genes and motif locations is provided in Table 7.

Chromatin Immunoprecipitation (ChIP)

The ChIP assays were performed using a modified protocol available at the worldwide web natureprotocols.com/2009/01/08/chromatin_immunoprecipitation_{—}2.php, which is hereinafter described. A 1/10 of chromatin solution was used as input DNA to determine DNA fragment sizes (0.3-1.0-kbp). The remaining chromatin solution was diluted 10-fold and divided into two aliquots; one was incubated with 10 μl of antibodies (anti-dimethyl-H3-Lys4, anti-dimethyl-H3-Lys9, anti-acetyl-H3-Lys9, all from Upstate Biotechnology, NY; or anti-CCA1), and the other incubated with protein beads. The immunoprecipitated DNA was amplified by semi-quantitative PCR using the primers designed from the conserved sequences of the CCA1, LHY, TOC1, and GI upstream of the ATG codon from both A. thaliana and A. arenosa loci (Table 4—shown below). Two independent experiments were performed and analyzed.

Electrophoretic Mobility Shift Assay (EMSA)

A CCA1 full-length cDNA was amplified from A. thaliana cDNA using a primer pair ATTB1_CCA1_F_XHO: 5′-GGGGACAAGTTTGTACAAAAAAGCAGGCTCCCTCGAGATGGAGACAAATTCGTC T-3′ (SEQ ID NO 13) and CCA1-R-Avr2-AttB2: 5′-GGGGACCACTTTGTACAAGAAAGCTGGGTCCCCTAGGTCATGTGGAAGCTTGAG TTTC-3′. (SEQ ID NO 14)

The cDNA was cloned into pDONR221 and validated by sequencing. The resulting insert was transferred by recombination into pET300/NT-DEST expression vector (Invitrogen Corp., Carlsbad, Calif.) and expressed in Escherichia coli Rosetta-gami B competent cells (Novagen, Madison, Wis.). Recombinant CCA1 protein was purified and subjected to EMSA in 6% native polyacrylamide gels using rCCA1 (10 fmoles) and ³²P-labeled double-stranded oligonucleotides (10 fmoles, Table 5). The cold probe (Cp) concentrations were 0 (-), 50 (5×), 100 (10×), 200 (20×), and 500 (50×) fmoles, respectively, according to a published protocol available at the worldwide web natureprotocols.com/2009/01/08/the_electrophoretic_mobility_s_{—}1.php.

Western Blot Analysis

Protein crude extracts were prepared from fresh leaves as previously described. The immunoblots were probed with anti-CCA1, and antibody binding was detected by ECL (Amersham, Piscataway, N.J.).

Results

In stable allotetraploids that were resynthesized by interspecific hybridization between A. thaliana and A. arenosa (FIG. 5), over 1,400 genes (>5% and up to 9,800 genes or ˜38%) were nonadditively expressed. Nonadditive expression indicates that the expression level of a gene in an allotetraploid is not equal to the sum of two parental loci (1+1≠2), leading to activation (>2), repression (<2), dominance, or overdominance. Many genes in energy and metabolism including photosynthesis and starch pathways are upregulated, coinciding with growth vigor in the allotetraploids. This morphological vigor is commonly observed, and phenotypic variation among allotetraploids is related to genetic and epigenetic mechanisms.

Among 128 genes upregulated in the allotetraploids, 86 (˜67%) each contains at least one CBS (AAAAATCT) (SEQ ID NO 12) or evening element (EE, AAAATATCT) (SEQ ID NO 11) within the ˜1,500-kbp upstream region (Table 7), which is significantly higher than all genes containing putative EE and CBS (˜15%, χ²=157 and P≦2.2e⁻¹⁶). These EE- and CBS-containing genes are likely the targets of CCA1 and LHY.

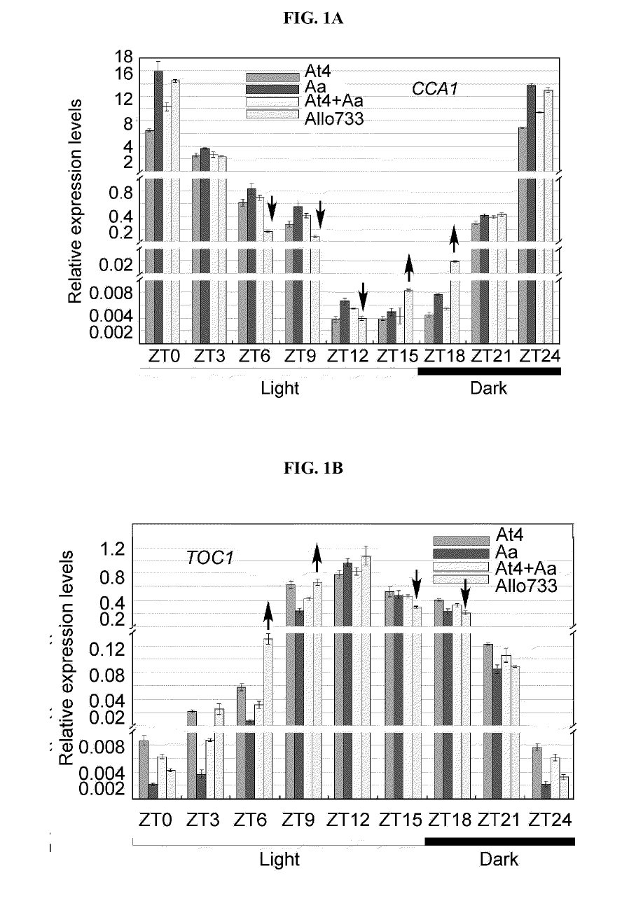

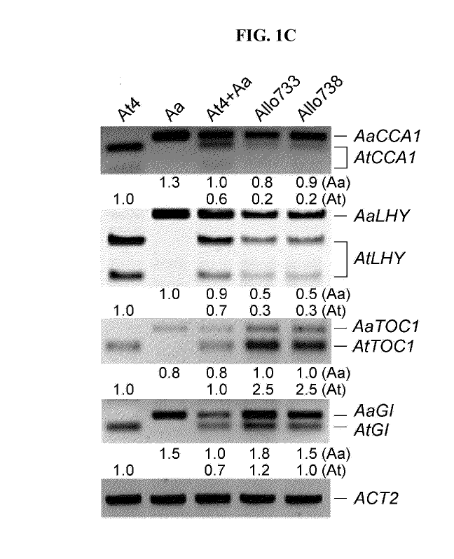

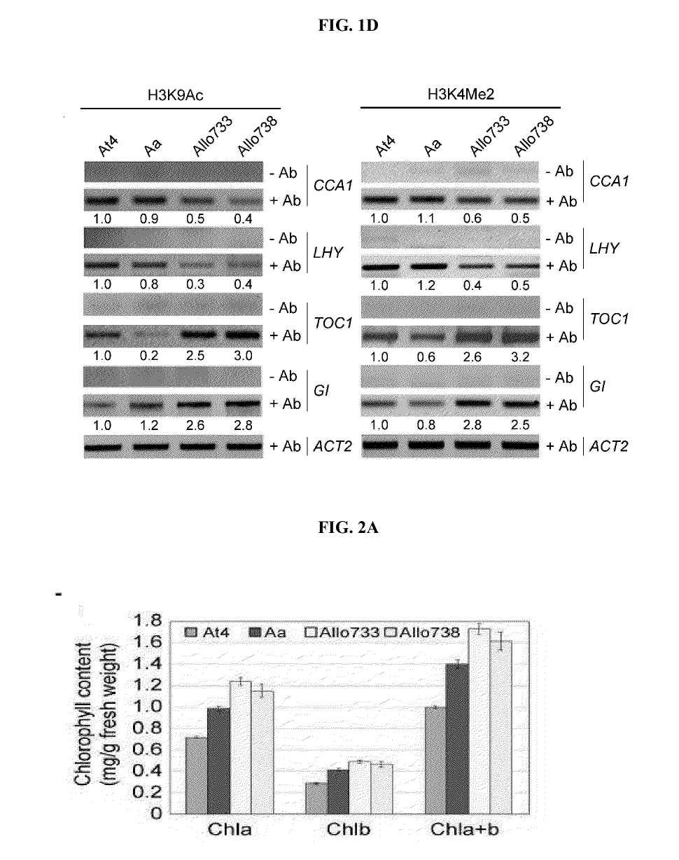

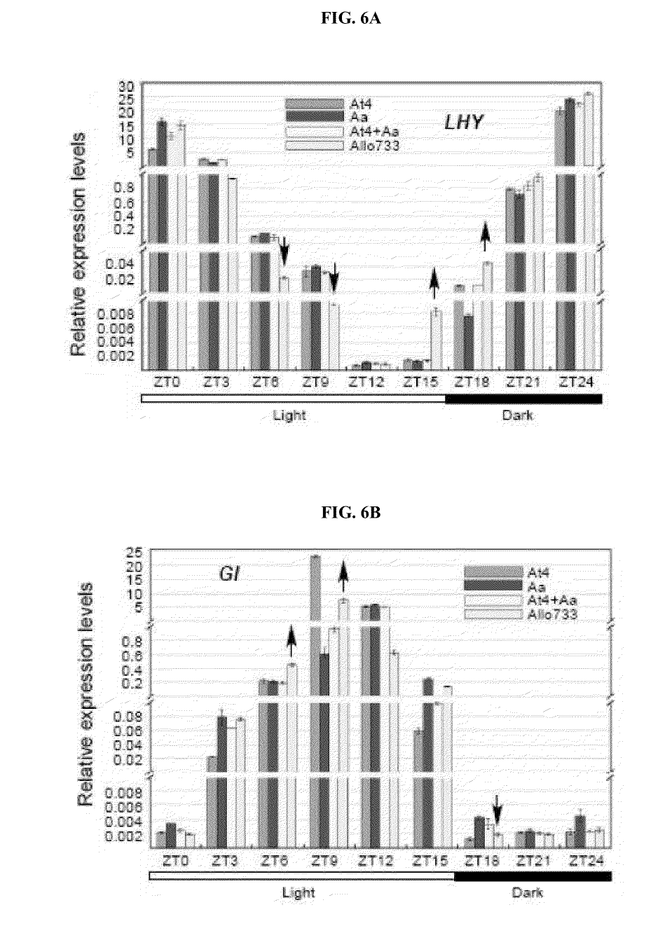

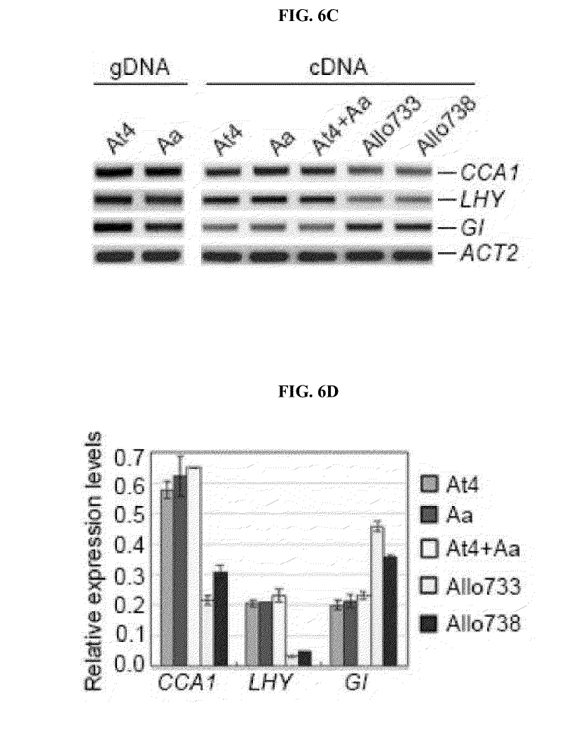

The present disclosure is based in part on the observation that CCA1 and LHY were repressed, and TOC1 and GI were upregulated, at noon in allotetraploids. As in the parents, both CCA1 and LHY displayed diurnal expression patterns in the allotetraploids (FIG. 1a and FIG. 6a). Table 1 is a table that shows the primer sequences of CCA1, LHY, TOC1 and GI used for quantitative RT-PCR analysis, according to the specific example embodiments of the present disclosure.

TABLE 1

| Locus | Name | Forward Primer | Reverse Primer |
|---|---|---|---|
| At2g46830 | CCA1 | 5′-CCTCGTCAGACACAGACTTCCA-3′ | 5′-CCGCAGTAGAATCAGCTCCAATA-3′ |
| | | (SEQ ID NO 15) | (SEQ ID NO 16) |
| At1g01060 | LHY | 5′-GGGACAAAGACTGCTGTTCAGAT-3′ | 5′-TTTGTGAAGAACTTTTGTGCATGA-3′ |
| | | (SEQ ID NO 17) | (SEQ ID NO 18) |
| At5g61380 | TOC1 | 5′-GTTGATGGATCGGGTTTCTC-3′ | 5′-TCATGACCCCATGCATACAG-3′ |
| | | (SEQ ID NO 19) | (SEQ ID NO 20) |
| At1g22770 | GI | 5′-TCGAGCAACTTCATCATCACAAA-3′ | 5′-GCTAATGGAGCTGGTGTCATACTG-3′ |
| | | (SEQ ID NO 21) | (SEQ ID NO 22) |
| At5g09810 | ACT2 | 5′-GTCTGTGACAATGGAACTGGAA-3′ | 5′-CTTTCTGACCCATACCAACCAT-3′ |
| | | (SEQ ID NO 23) | (SEQ ID NO 24) |

Their expression peaked at dawn (ZT0), decreased 6 hours after dawn (ZT6), and continued declining until dusk (ZT15). CCA1 and LHY were expressed 2-4-fold lower in the allotetraploids than the mid-parent value (MPV) at ZT6-12 and higher than the MPV at dusk (ZT15). TOC1 and GI expression was inversely correlated with CCA1 and LHY expression (FIG. 1b and FIG. 6b), suggesting feedback regulation in the allotetraploids as in the diploids. However, TOC1 and GI expression fluctuated in the allotetraploids, indicating that other factors may be involved. The expression changes of these genes from noon to dusk in the allotetraploids may alter the amplitude but not the phase of circadian clock, as they quickly gained the expression levels similar to MPV after dusk (ZT18-24).

To determine how CCA1 and LHY expression was repressed, expression patterns of A. thaliana and A. arenosa loci in the allotetraploids were examined using RT-PCR and cleaved amplified polymorphic sequence (CAPS) analyses that are discriminative of locus-specific expression patterns. While A. thaliana and A. arenosa loci were equally expressed in respective parents, in two allotetraploids A. thaliana CCA1 (AtCCA1) expression was down-regulated ˜3-fold, and A. arenosa CCA1 (AaCCA1) expression was slightly reduced (FIG. 1c). Similarly, AtLHY expression was dramatically reduced (˜3.3-fold), whereas AaLHY expression was decreased ˜2-fold in the allotetraploids. Conversely, AtTOC1 and AtGI loci were upregulated in the allotetraploids. The data suggest that A. thaliana genes are more sensitive to expression changes in the allotetraploids probably through cis- and trans-acting effects and chromatin modifications as observed in other loci.

Table 2 shows primer sequences of CCA1, LHY, TOC1 and GI for RT-PCR and CAPS analysis, according to the specific example embodiments of the present disclosure.

TABLE 2

| Locus | Name | Forward Primer | Reverse Primer |
|---|---|---|---|
| At2g46830 | CCA1 | 5′-GGATCGGTTATATTGGAGCTGA-3′ | 5′-AACACCTGAGAGTGATGCAAAG-3′ |
| | | (SEQ ID NO 25) | (SEQ ID NO 26) |
| At1g01060 | LHY | 5′-TTGATCTTGCAGAGCTGTGTTTTG-3′ | 5′-CTTGGTGGGCTTCTCATGGACT-3′ |
| | | (SEQ ID NO 27) | (SEQ ID NO 28) |
| At5g61380 | TOC1 | 5′-GCGCCAGCTATCCACATTCC-3′ | 5′-CTTGGTTCATGACCCCATGC-3′ |
| | | (SEQ ID NO 29) | (SEQ ID NO 30) |
| At1g22770 | GI | 5′-ATCACTGATCCATGGAGACAAA-3′ | 5′-GAGAAATCCTTCGCATTTTGA-3′ |
| | | (SEQ ID NO 31) | (SEQ ID NO 32) |
| At5g09810 | ACT2 | 5′-CTCATGAAGATTCTCACTGAG-3′ | 5′-ACAACAGATAGTTCAATTCCCA-3′ |
| | | (SEQ ID NO 33) | (SEQ ID NO 34) |

Chromatin changes in the upstream regions (˜250-bp) of CCA1, LHY, TOC1, and GI (Table 4) were examined using antibodies against histone H3-Lys9 acetylation (H3K9Ac) and H3-Lys4 dimethylation (H3K4Me2), two marks for gene activation. H3K9Ac and H3K4Me2 levels in the CCA1 and LHY promoters were 2-3-fold lower in the allotetraploids than that in A. thaliana and A. arenosa (FIG. 1d), consistent with CCA1 and LHY repression. Likewise, TOC1 and GI upregulation correlated with increased levels of H3K9Ac and H3K4Me2. Changes in H3K9Me2, a heterochromatic mark, were undetectable (data not shown). These data suggest that diurnal expression changes of LHY, CCA1, TOC1, and GI are associated with euchromatic histone marks. Alternatively, autonomous pathways and other factors such as ELF4 may mediate TOC1 and GI expression.

In summary, FIG. 1 shows locus-specific and chromatin regulation of circadian clock genes in allotetraploids. FIG. 1a shows a qRT-PCR analysis of CCA1 expression (n=3, ACT2 as a control) in a 24-hour period (light/dark cycles) starting from dawn (ZT0, 6 am) (arrows indicate up- and down-regulation, respectively). FIG. 1b shows a qRT-PCR analysis of TOC1 expression (n=3). FIG. 1c shows the repression of A. thaliana CCA1 and LHY and upregulation of A. thaliana TOC1 and GI in allotetraploids. RT-PCR products were digested with AvaII (CCA1), AflIII (LHY), SspI (TOC1), and SpeI (GI). FIG. 1d shows the ChIP analysis of CCA1, LHY, TOC1, and GI using antibodies against H3K9Ac and H3K4Me2 (n=2). -Ab: no antibodies.

Table 3 shows primer sequences of CCA1, LHY, TOC1 and GI putative promoters for ChIP analysis, according to the specific example embodiments of the present disclosure.

TABLE 3

| Locus | Name | Forward Primer | Reverse Primer |
|---|---|---|---|
| At2g46830 | CCA1 | 5′-GGAGAAATCTCAGCCACTATAA-3′ | 5′-AACTCGTGGCCTAGAATACA-3′ |
| | | (SEQ ID NO 35) | (SEQ ID NO 36) |
| At1g01060 | LHY | 5′-GCTGGAACAGCACCAAGGGTAT-3′ | 5′-CTGGCACCGTACCCACTTGTTT-3′ |
| | | (SEQ ID NO 37) | (SEQ ID NO 38) |
| At5g61380 | TOC1 | 5′-AAACGAAACGAAGCCGAATCC-3′ | 5′-GGTCAAATTATGAGACGCGAAA-3′ |
| | | (SEQ ID NO 39) | (SEQ ID NO 40) |
| At1g22770 | GI | 5′-ATGGTAATGGCGCATAAA-3′ | 5′-CAAATGATTCGGGAAAA-3′ |
| | | (SEQ ID NO 41) | (SEQ ID NO 42) |
| At5g09810 | ACT2 | 5′-CGTTTCGCTTTCCTTAGTGTTAGCT-3′ | 5′-AGCGAACGGATCTAGAGACTCACCTTG-3′ |
| | | (SEQ ID NO 43) | (SEQ ID NO 44) |

To test downstream effects of CCA1 and LHY repression, the expression of two subsets of EE/CBS-containing genes were examined (Table 6). One subset consists of the genes encoding protochlorophyllide (pchlide) oxidoreductases a and b, PORA and PORB, that mediate the only light-requiring step in chlorophyll biosynthesis in higher plants. PORA and PORB are strongly expressed in seedlings and young leaves, and upregulation of PORA and PORB increases chlorophyll a and b content. Both PORA and PORB were upregulated in the allotetraploids (FIG. 2c). The total chlorophyll content in both allotetraploids was ˜60% higher than in A. thaliana and ˜15% higher than in A. arenosa (FIG. 2a). Chlorophyll a increased more than chlorophyll b, and the allotetraploids accumulated ˜70% more chlorophyll a than A. thaliana.

The other subset of EE/CBS-containing genes encodes enzymes in starch metabolism and sugar transport, many of which show strong diurnal rhythmic expression patterns. Starch metabolism involves the genes encoding AMY3, BAM1, 2 and 3, DPE1 and 2, GTR, GWD1 and 3, ISA1, 2 and 3, LDA, MEX1, and PHS1 and 2 (FIG. 2b). Many contained an evening element or CBS (Table 6) and were upregulated 1.5-4-fold in allotetraploids (FIG. 2e), when CCA1 and LHY were down-regulated (FIGS. 1a and 1c). MTR, BAM3 and BAM4, which all lacked an evening element or CBS, showed little expression changes, suggesting that their expression is independent of clock regulation or undergoes post-transcriptional regulation.

Table 4 shows primer sequences of the genes involved in photosynthesis and starch degradation for RT-PCR analysis

TABLE 4

| Locus | Protein | Symbol | Forward Primer | Reverse Primer |
|---|---|---|---|---|
| At5g54190 | protochloropbyllide | PORA | 5′-AAACCATTTGGGCCACTTT | 5′-CAAGTCTTTTCCCAGCCTCT |
| | reductase | | CTT-3′ (SEQ ID NO 45) | GA-3′ (SEQ ID NO 46) |
| At4g27440 | protochloropbyllide | PORB | 5′-ACCAAATCAAATCCGAACA | 5′-GGCTCTTTAGCTGTCGGGAA |
| | reductase b | | TGG-3′ (SEQ ID NO 47) | AT-3′ (SEQ ID NO 48) |
| AtlglO760 | α-glucan, water | GWD1 | 5′-TTCCTCCTTTGCTTTGGCGT | 5′-TCCAGTGGACGGGAGGAAAA-3′ |
| | dikinase | | A-3′ (SEQ ID NO 49) | (SEQ ID NO 50) |
| At5g26570 | Phosphoglucan | GWD3 | 5′-GCCATTGTTGCAGCTCTCCT | 5′-TTCCAACTCACAAACCCATCC |
| | | | TT-3′ (SEQ ID NO 51) | A-3′ (SEQ ID NO 52) |
| | water dikanse | | | |
| Atlg69830 | alpha-amylase- | AMY3 | 5′-CGGTGGAGGTAACCACAGA | 5′-AAACTGGCTGCGGAGGCATA-3′ |
| | like 3 | | ACA-3′ (SEQ ID NO 53) | (SEQ ID NO 54) |
| At2g39930 | isoamylase 1 | ISA1 | 5′-GCCATGTTTGGCATGTGTT | 5′-AAAACCTCGCACATGCATTTC |
| | | | CTT-3′ (SEQ ID NO 55) | A-3′ (SEQ ID NO 56) |
| Atlg03310 | isoamylase 2 | ISA2 | 5′-TCCCAAGACTCACAAACCC | 5′-TGAAGCATGCCAAACATCACC |
| | | | ACA-3′ (SEQ ID NO 57) | T-3′ (SEQ ID NO 58) |
| At4g09020 | isoamylase 3 | ISA3 | 5′-GCATCTACAATGACGGAGA | 5′-GCCATTCTCCAGGACCATCAA |
| | | | CGAAA-3′ (SEQ ID NO 59) | C-3′ (SEQ ID NO 60) |
| At5g04360 | pullulanase | LDA1 | 5′-CGCCGCTGATTTTAATCTTC | 5′-TCGATTCCATCTTCGCTGAAT |
| | 1/Limit dextrinase | | GAT-3′ (SEQ ID NO 61) | G-3′ (SEQ ID NO 62) |
| At5g64860 | D-Enzyme | DPE1 | 5′-CCAAAACCCTGCAAATCCT | 5′-AAAGGCAGTGGCAGAAAGTTC |
| | | | CTG-3′ (SEQ ID NO 63) | G-3′ (SEQ ID NO 64) |
| At2g40840 | Transglucosidase | DPE2 | 5′-GCAGCAGCAGAATATGCAA | 5′- CGCCACCAGGCATAGTTGT |
| | | | GGA-3′ (SEQ ID NO 65) | C-3′ (SEQ ID NO 66) |
| Atlg29320 | Plastidicglucan | PHS1 | 5′-ATTATCCGGCTTGGGGTTA | 5′-TCGGAAGGAGCTTTTGTTGAC |
| | phosphorylase | | TGG-3′ (SEQ ID NO 67) | C-3′ (SEQ ID NO 68) |
| Atlg46970 | Cytosolicglucan | PHS2 | 5′-AGCTGCCTCTTGTATTCGTG | 5′-TGAATGGCGTTGCTCAGTTAC |
| | phosphorylaes | | GA-3′ (SEQ ID NO 69) | A-3′ (SEQ ID NO 70) |
| Atlg23920 | β-Amylase 1 | BAM1 | 5′-GGTGAATCGAAGAAAGGC | 5′-CATGGTTCCTTCTTCCCCACT |
| | | | GATG-3′ (SEQ ID NO 71) | G-3′ (SEQ ID NO 72) |
| At4g00490 | β-Amylase 2 | BAM2 | 5′-TTTTGGGGCAGAGGACCTG | 5′-GAGCCTCTGGGAAATCCTCATG |
| | | | ATA-3′ (SEQ ID NO 73) | T-3′ (SEQ ID NO 74) |
| At4g17090 | β-Amylase 3 | BAM3 | 5′-GCCTTCAAATCGTTCACGG | 5′-AAAGGAGCTGGTGTGGAAGGT |
| | | | AAG-3′ (SEQ ID NO 75) | G-3′ (SEQ ID NO 76) |
| At5g55700 | β-Amylase 4 | BAM4 | 5′-GGTGTTCATGGAATCGCAG | 5′-GCCACCGAACAAAGGAAGTTG |
| | | | TTG-3′ (SEQ ID NO 77) | A-3′ (SEQ ID NO 78) |
| Atlgl6150 | Glucose | GTR | 5′-TTTGCGTTTCAGAGACGGA | 5′-AGCACCAAGACAAGCAACACC |
| | transporter | | CCT-3′ (SEQ ID NO 79) | A-3′ (SEQ ID NO 80) |
| At5g17520 | maltose | MTA | 5′-TCCCACAGTTGCCACACAG | 5′-GGTGGAGCAAACATTCCGTTT |
| | transporter | | TTT-3′ (SEQ ID NO 81) | C-3′ (SEQ ID NO 82) |
| At5g09810 | Actin | ACT2 | 5′-CTCATGAAGATTCTCACTG | 5′-ACAACAGATAGTTCAATTCCC |
| | | | AG-3′ (SEQ ID NO 83) | A-3′ (SEQ ID NO 84) |

In summary, FIG. 2 shows an increase in chlorophyll content and upregulation of the genes involved in chlorophyll and starch biosynthesis in allotetraploids. Table 6 depicts locations of CCA1 binding site (CBS) or evening element (EE) in the downstream genes (Table 7). Lower-case letter: nucleotide variation. FIG. 2a depicts the increase of chlorophyll (a, b, and total) content in the allotetraploids (n=3). FIG. 2b depicts starch metabolic pathways (modified from that of 26) in the chloroplast (circled) and cytoplasm. FIG. 2 depicts the upregulation of PORA and PORB in the allotetraploids at ZT6 (n=2). gDNA: Genomic PCR. FIG. 2d depicts the upregulation of starch metabolic genes in allotetraploids (n=2) at ZT6.

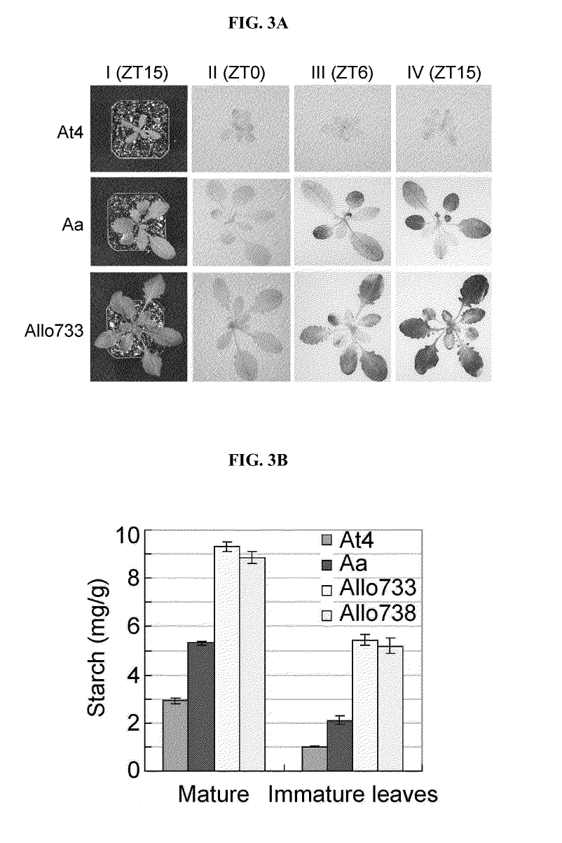

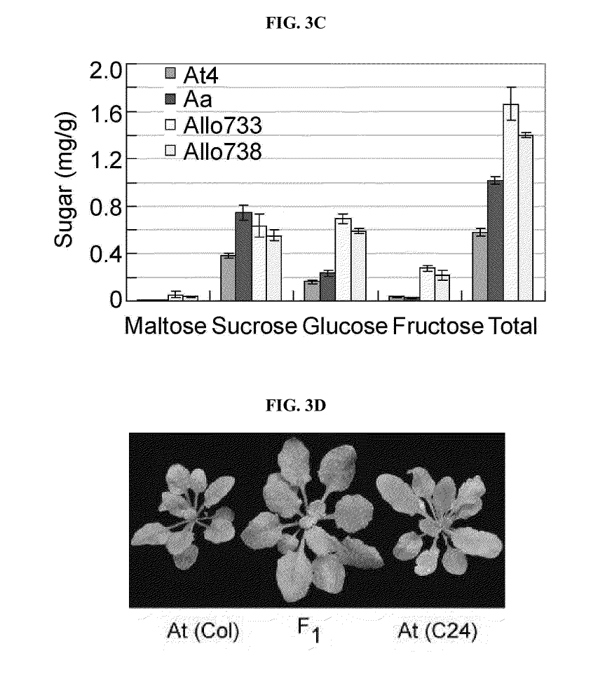

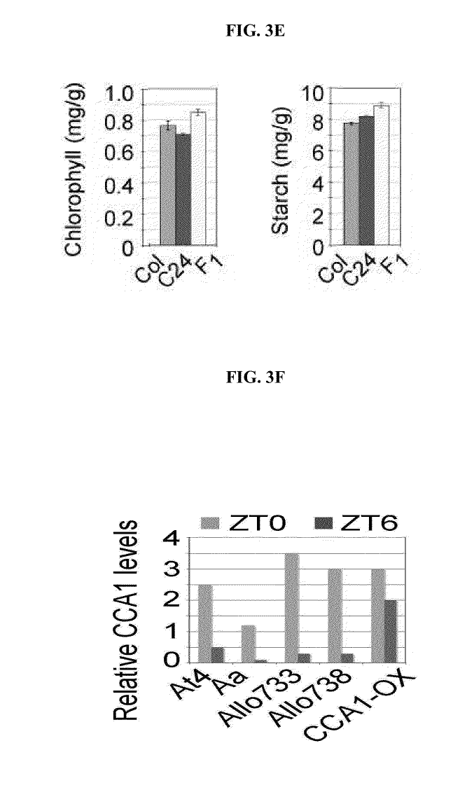

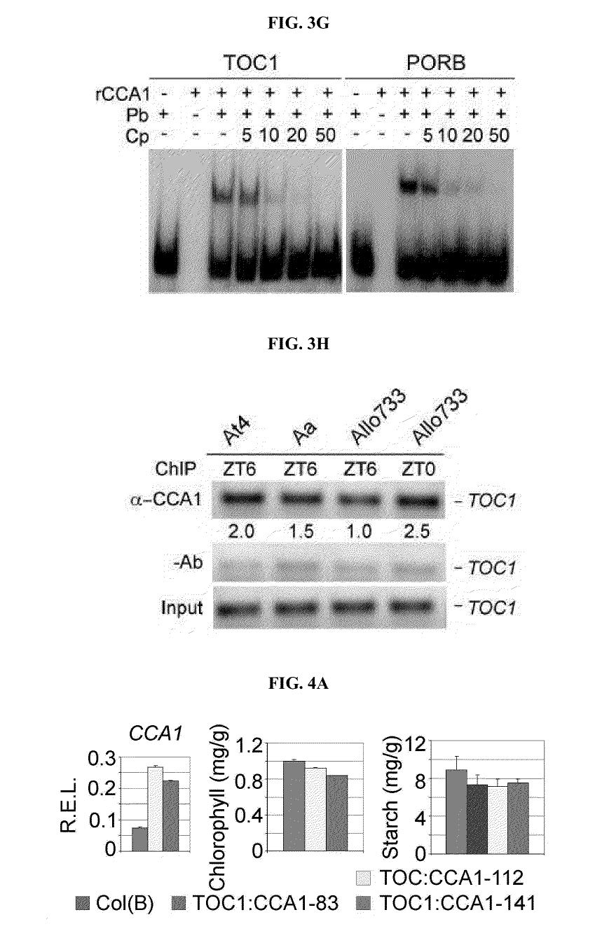

Allotetraploids accumulated more starch than the parents in both mature and immature leaves using iodine-staining (FIG. 3a) and quantitative assays (FIG. 3b). In the mature leaves, allotetraploids accumulated starch 2-fold higher than A. thaliana and 70% higher than A. arenosa. In the immature leaves, allotetraploids contained 4-fold higher starch than A. thaliana and 50-100% higher sugar content than the parents (FIG. 3c), mainly due to increases in glucose and fructose content, suggesting high rates of starch and sugar accumulation in young leaves. The sucrose content in allotetraploids was similar to A. arenosa but higher than in A. thaliana in immature leaves and similar among all lines tested in mature leaves (data not shown), indicating rapid transport and metabolism of sucrose especially in the mature leaves. Together, chlorophyll, starch, and sugar amounts were consistently high in the allotetraploids.

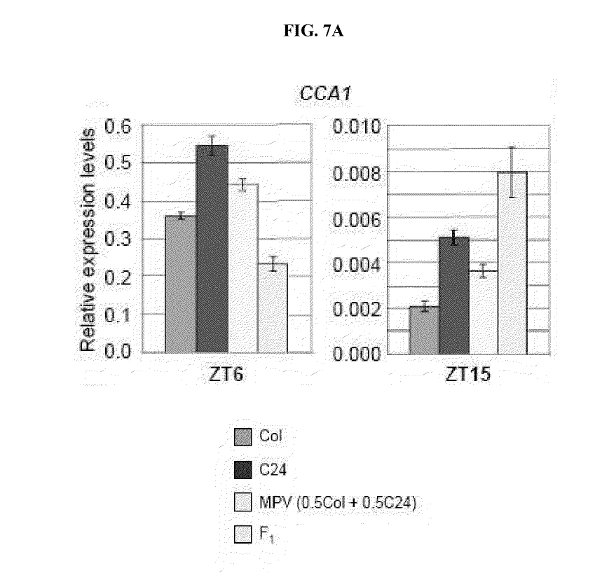

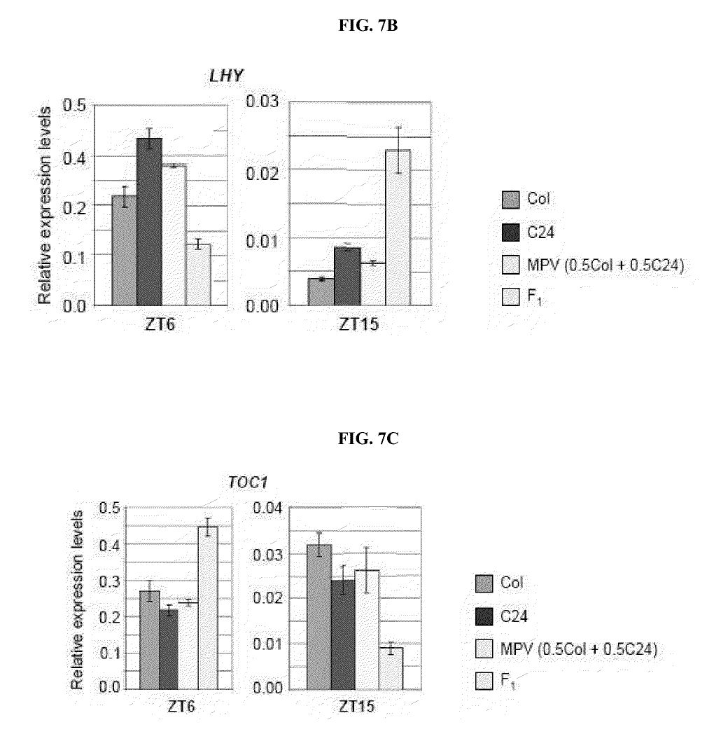

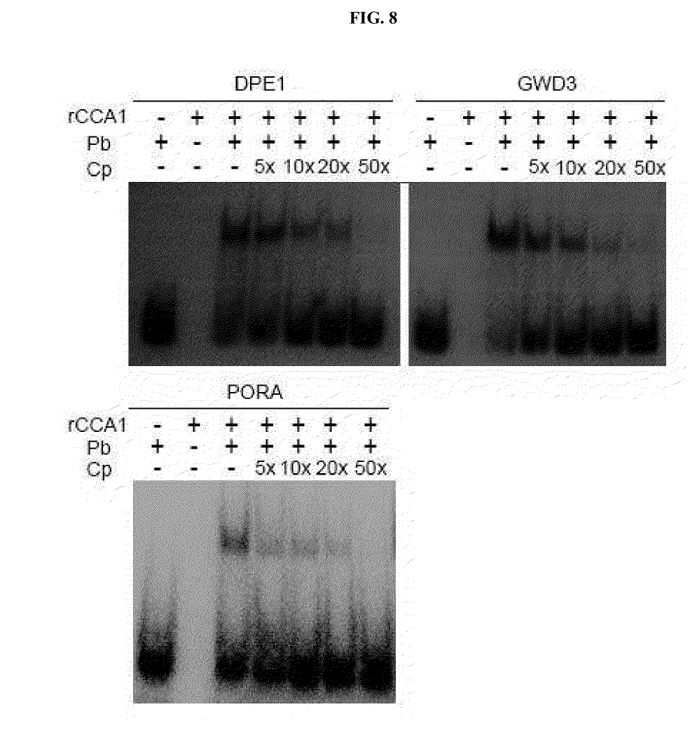

It was further tested if circadian clock regulation was altered in F₁ hybrids as in the interspecific hybrids and alloptetraploids. At ZT6 (noon), CCA1 and LHY were repressed ˜2-fold, whereas TOC1 was upregulated ˜2-fold in the F₁ hybrids relative to the parents (C24 and Columbia) (FIG. 7). At ZT15, CCA1 and LHY were upregulated, whereas TOC1 was repressed in the hybrids. The F₁ hybrids displayed morphological vigor (FIG. 3d) and contained ˜12% more total chlorophylls and ˜10% more starch than the higher parent (FIG. 3e).

To determine how CCA1 regulates downstream genes and output traits, CCA1 function was examined in the allotetraploids and their parents. CCA1 protein levels in these lines were high at dawn (ZT0) and low at noon (ZT6) (FIG. 3f), corresponding to the CCA1 transcript levels (FIG. 1a). CCA1 levels were constantly high in A. thaliana constitutive CCA1-overexpression (CCA1-OX) lines. Electrophoretic mobility shift assay (EMSA) indicated specific binding of recombinant CCA1 to EE-containing fragments of the target genes TOC1, PORB, PORA, DPE1, and GWD3 (FIG. 3g, FIG. 8 and Table 5). Using antibodies against CCA1 in chromatin immunoprecipitation (ChIP) assays, it was further demonstrated that endogenous CCA1 in the TOC1 promoter was ˜2.5-fold lower at ZT6 (noon) than at ZT0 (dawn) (FIG. 3h), which is inversely correlated with TOC1 expression levels that were higher at noon than at dawn (FIG. 1b).

Table 5 shows the oligonucleotides used for electrophoretic mobility shift assays, according to the specific example embodiments of the present disclosure.

TABLE 5

| | | Evening | | |
|---|---|---|---|---|
| Gene | Locus | element | Oligos | Location |
| GWD3 | At5g26570 | AAAAaATC | 5′-cacaaacaAAAAAATCTcttatcac-3′ | −35 to −10 |
| | | T | (SEQ ID NO 85) | |
| | | | 5′-gtgataagAGATTTTTTtgtttgtg-3′ | |
| | | | (SEQ ID NO 86) | |
| DPE1 | At5g64860 | AAAATAT | 5′-agagcaacAAAATATCTcgactgtt-3′ | −87 to −62 |
| | | CT | (SEQ ID NO 87) | |
| | | | 5′-aacagtcgAGATATTTTgttgctct-3′ | |
| | | | (SEQ ID NO 88) | |
| PORA | At5g54190 | AAAATAT | 5′-tatacattAAAATATCTactgacag-3′ | −229 to −204 |
| | | CT | (SEQ ID NO 89) | |
| | | | 5′-ctgtcagtAGATATTTTaatgtata-3′ | |
| | | | (SEQ ID NO 90) | |
| PORB | At4g27440 | AAAATAT | 5′-attaaaatAAAATATCTaaggagaa-3′ | −252 to −227) |
| | | CT | (SEQ ID NO 91) | |
| | | | 5′-ttctccttAGATATTTTattttaat-3′ | |
| | | | (SEQ ID NO 92) | |
| TOC1 | At5g61380.1 | AAAATAT | 5′-acacaaaaAAAATATCTaatcacag-3′ | −47 to −22 |
| | | CT | (SEQ ID NO 93) | |
| | | | 5′-ctgtgattAGATATTTTttttgtgt-3′ | |
| | | | (SEQ ID NO 94) | |

* Location was counted upstream of the ATG codon, and EE sites are shown in upper-case letters.

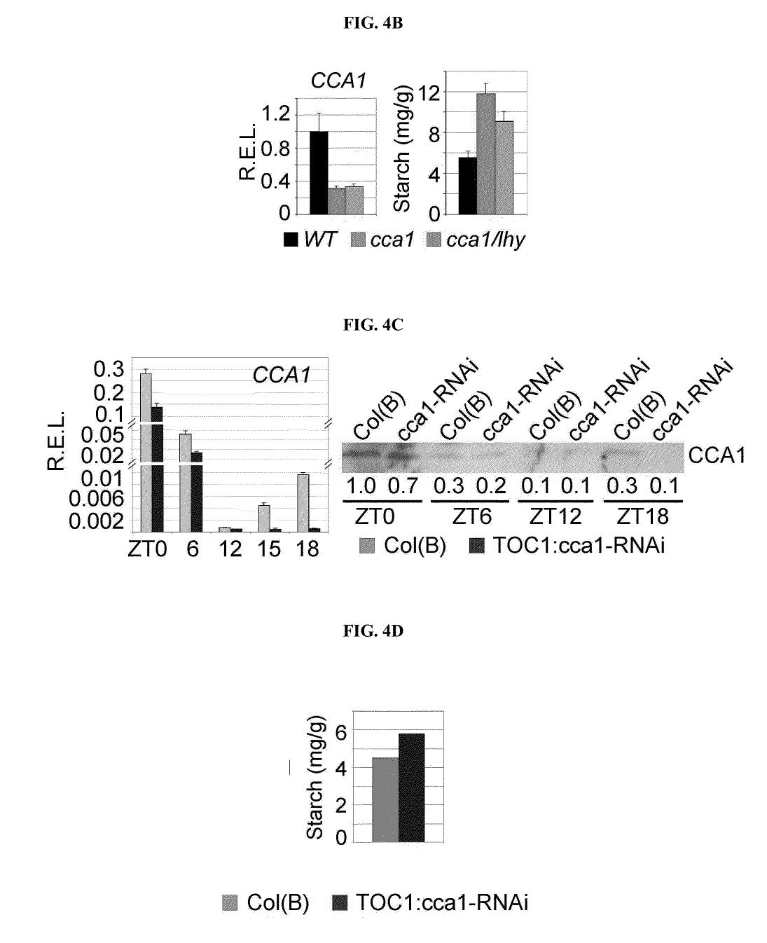

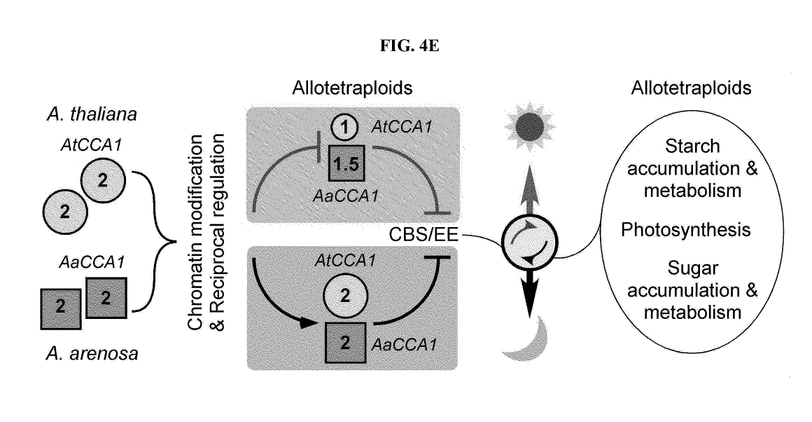

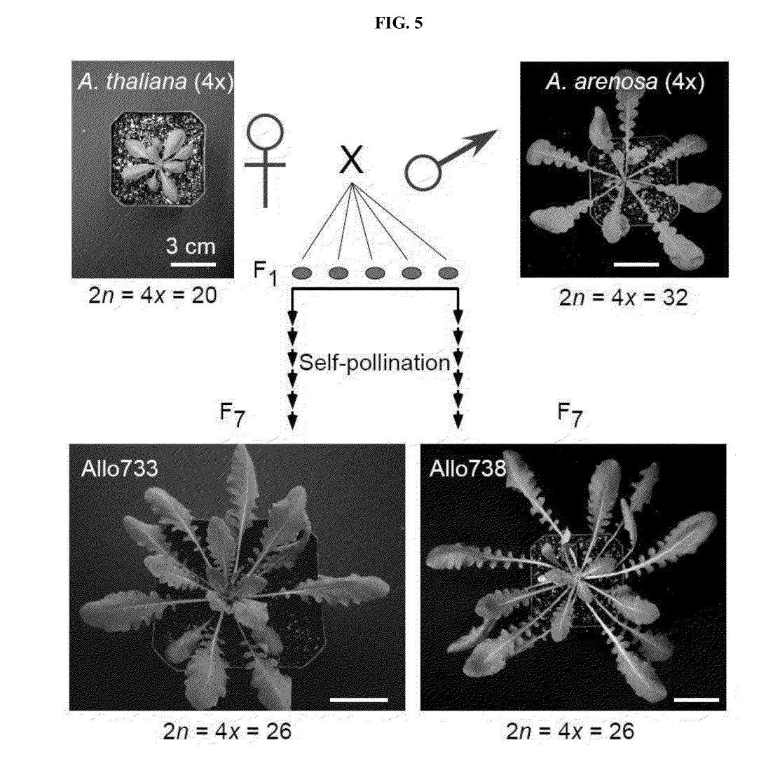

In summary, FIG. 4 shows the role of CCA1 in growth vigor in allotetraploids and hybrids. FIG. 4a depicts relative expression levels of CCA1 (ZT6, left) and reduced chlorophyll (ZT9, middle) and starch (ZT15, right) accumulation in TOC1:CCA1 lines (n=3) (FIG. 8). Col(B): Columbia transformed with basta gene. FIG. 4b depicts reduced CCA1 expression (ZT6, left) and increased starch content (ZT15, right) in cca1-11 and cca1-11 lhy-21 mutants (n=3). WT: Wassilewskija (Ws) or Col. FIG. 4c depicts decreased expression of CCA1 mRNA (right, n=3) and protein (right, n=2) (ZT0-18, T2) in TOC1:cca1-RNAi transgenic plants. FIG. 4d depicts increased starch content in TOC1:cca1-RNAi lines (ZT15, n=2). FIG. 4e depicts a model for growth vigor and increased biomass. Chromatin-mediated changes in internal clock regulators (e.g., AtCCA1) in allotetraploids lead to up- and down-regulation and normal oscillation of gene expression and output traits (photosynthesis, starch and sugar metabolism) at noon (sun) and dusk (moon).

In summary, FIG. 5 shows the Arabidopsis allotetraploids (2n=4x=26) were resynthesized by interspecific hybridization between A. thaliana autotetraploid (At4, 2n=4x=20) and pollen donor A. arenosa (Aa, 2n=4x=32), an outcrossing tetraploid. The resulting allotetraploids were self-pollinated for 7 generations to generate stable allotetraploids that contain complete sets of A. thaliana and A. arenosa chromosomes. Seedling of A. thaliana, A. arenosa, and two allotetraploid lines (Allo733 and Allo738, F7) at similar developmental stages (before bolting) are shown. Scale bars indicate 3 cm.

In summary, FIG. 6 shows the expression of circadian clock regulators (LHY and GI) in a 24-hour period using zeitgeber time (ZT) starting from dawn (ZT0). FIG. 6a depicts Quantitative RT-PCR (qRT-PCR) analysis of LHY expression. Relative expression levels were calculated using ACT2 as a control. The standard deviations were calculated from three biological replications. Downward and upward arrows indicate down- and upregulation of CCA1 expression in the resynthesized allotetraploid (Allo733), respectively. At4: A. thaliana autotetraploid; Aa: A. arenosa; and At4+Aa: mid-parent using an equal mixture of RNAs from At4 and Aa. Light and dark periods are indicated below the graph. The gaps in the bars indicate large changes in R.E.L. FIG. 6b depicts qRT-PCR analysis of GI expression. The labels and abbreviations are the same as in FIG. 6a. The standard deviations were calculated from three biological replications. FIG. 6c depicts genomic and RT-PCR analysis of CCA1, LHY, TOC1, and GI in A. thaliana (At4), A. arenosa(Aa), mid-parent (At4+Aa), and two allotetraploid lines (Allo733 and Allo738). FIG. 6d depicts qRT-PCR analysis of CCA1, LHY, and GI in At4, Aa, At4+Aa, and two allotetraploids at noon (ZT6).

In summary, FIG. 7 shows the expression of circadian clock regulators (CCA1, LHY and TOC1) in Arabidopsis thaliana hybrids (F1) and their parents (C24 and Columbia, Col) at zeitgeber time 6 and 15 (ZT6 and ZT15, ZT0=dawn). FIG. 7a depicts qRT-PCR analysis of CCA1 expression at ZT6 and ZT15. MPV: mid parent value, an equal mixture of RNAs from Col and C24. FIG. 7b depicts qRT-PCR analysis of LHY expression at ZT6 and ZT15. FIG. 7c depicts qRT-PCR analysis of TOC1 expression at ZT6 and ZT15. The labels and abbreviations in FIG. 7b and FIG. 7c are the same as in FIG. 7a. Relative expression levels were calculated using ACT2 as a control. The standard deviations were calculated from three biological replications.

FIG. 8 summarizes the results of the electrophoretic mobility shift assay (EMSA) showing competitive binding of recombinant CCA1 to DPE1, GWD3, and PORA promoter fragments. The concentration of 32P-labeled probe (Pb) and recombinant CCA1 (rCCA1) was 10 fmoles each. The cold or competitive probe (Cp) concentrations were 0 (-), 50 (5×), 100 (10×), 200 (20×), and 500 (50×) fmoles, respectively.

FIG. 9 is a characterization of CCA1 overexpression lines driven by 35S and TOC1 promoters. FIG. 9a depicts ectopic expression of CCA1 under the control of 35S and TOC1 promoters. Typical plants prior to flowering were shown. Col: A. thaliana Columbia ecotype. Col(B): Col plants transformed with basta gene. CCA1-OX: constitutive CCA1 overexpression line (Wang et al. 1998); TOC1:CCA1-200, 112, and 83: three transgenic plants that ectopically expressed CCA1 driven by TOC1 promoter. Top panel: Col (B) and TOC1:CCA1 lines after spraying with basta (100 mg/L). FIG. 9b depicts a ProTOC1:CCA1 construct. Arrows indicate the primer pair of F-5′-TTGGTTTCTGATGGTTTGGTCTGA-3′ (SEQ ID NO 95) and R-5′-CGCTTGACCCATAGCTACACCTTT-3′ (SEQ ID NO 96). Genotyping TOC1:CCA1 transgenic plants. Among 36 plants, five (4, 7, 8, 10, and 30) did not contain the transgene. FIG. 9c depicts reduced chlorophyll content in the CCA1-OX line and TOC1:CCA1 transgenic plants at ZT9. FIG. 9d depicts decreased starch content in the leaves of TOC1:CCA1 transgenic lines at ZT6. Unless noted otherwise, standard deviations were calculated from three biological replications.

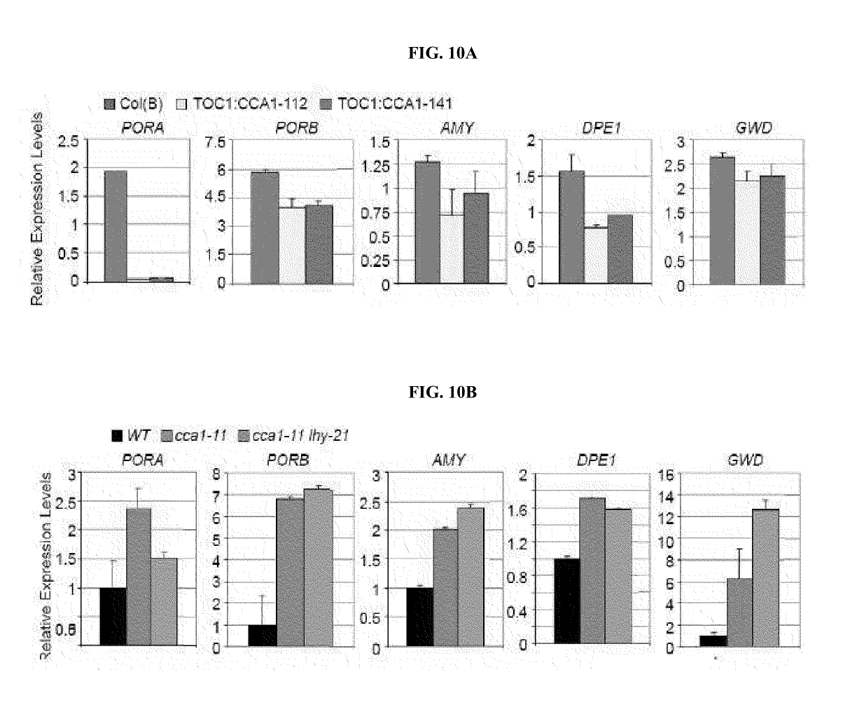

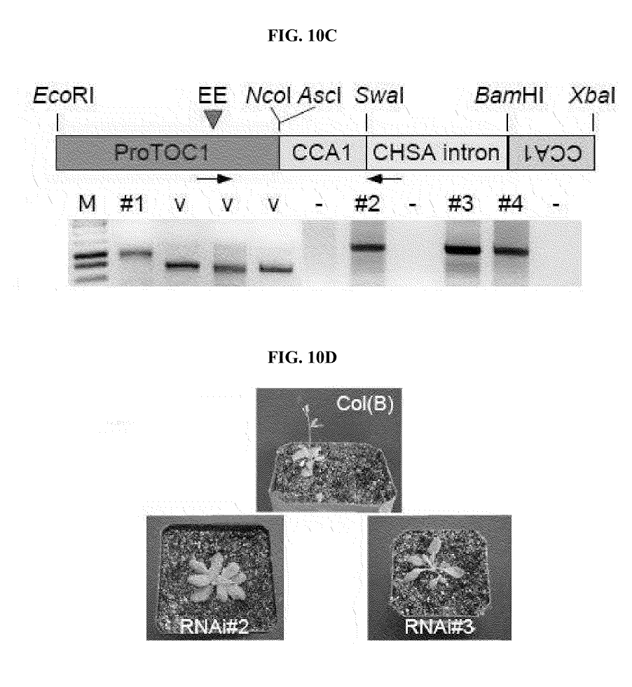

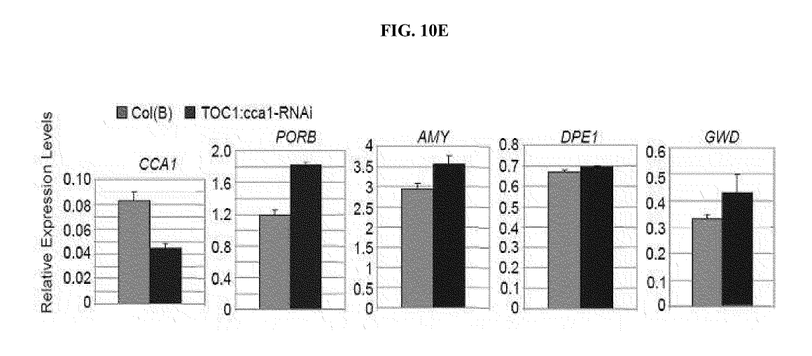

FIG. 10 shows the expression of downstream genes (PORA, PORB, AMY, DPE1, and GWD) in TOC1:CCA1 transgenics, cca1 and cca1 lhy mutants, and TOC1:cca1-RNAi lines. FIG. 10a depicts the down regulation of downstream genes (PORA, PORB, AMY, DPE1, and GWD) at ZT15 in transgenic plants (#112 and #141) that overexpressed CCA1 under the control of TOC1 promoter. Col(B): Transgenic A. thaliana (Columbia) plants containing a plasmid vector with the basta gene. FIG. 10b depicts the upregulation of downstream genes (PORA, PORB, AMY, DPE1, and GWD) at ZT6 in cca1-11 and cca1-11 lhy-21 mutants. WT: wild-type (A. thaliana ecotype Wassilewskija or Ws). ACT2 was used as a control. Unless noted otherwise, standard deviations were calculated from three biological replications. GWD: glucan-water dikinase; AMY: alpha-amylase; DPE: isproportionating enzyme. FIG. 10c depicts a ProTOC1:cca1-RNAi construct (Top panel) that was made from pFGC5941 by replacing the 35S promoter with the ProTOC1 promoter and using two subsequent steps of cloning 250-bp CCA1 fragments using BamHI and XbaI followed by AscI and SwaI. The resulting construct (pTOC1:cca1-RNAi) was used to transform A. thaliana Columbia. CHSA: chalcone synthase A gene (a 1,353-bp fragment). EE: evening element. The bottom panel of FIG. 10c depicts a subset of genotyping data shows four positive TOC1:cca1-RNAi lines (#1-4), three transgenics with vector only (v), and three nontransgenics (-). M: DNA size marker. The cca1 transgene fragment that is slightly larger than the vector fragment. The primer pair for cca1 transgene genotyping (indicated by arrows below the diagram) is FpTOC1:CCA1: 5′-TTGGTTTCTGATGGTTTGGTCTGA-3′ (SEQ ID NO 97) and Rintron: 5′-GAACCCGTTTGGGTGAGCTTAAAAGTGG-3′ (SEQ ID NO 98), and the primer pair for vector transgene genotyping is Fp35S 5′-AAGGGATGACGCACAATCCCACTATCC-3′ (SEQ ID NO 99) and Rintron. FIG. 10d shows images of TOC1:cca1-RNAi lines. Under long-day conditions, some TOC1:cca1-RNAi lines flowered early, while others flowered late (shown) relative to the control, Col(B). FIG. 10e depicts expression of CCA1 and downstream genes. CCA1 expression was repressed, whereas expression of PORB, AMY, DPE1, and GWD was induced at ZT15. Three transgenic plants were used as three replications in gene expression analysis, which may overestimate but not underestimate the variation.

These data collectively suggest that CCA1 directly affects TOC1 and downstream genes in clock regulation, photosynthesis, and starch metabolism. Clock dependent upregulation of output genes may lead to growth vigor. Indeed, overexpressing PORA and PORB increases chlorophyll content, seedling viability, and growth vigor in A. thaliana, while mutants of starch metabolic genes display reduced starch content and growth vigor. If CCA1 repression promotes growth, CCA1 overexpression would reduce growth vigor in diploids. Indeed, TOC1:CCA1 transgenic plants expressing CCA1 under the clock-regulated TOC1 promoter (FIG. 9) displayed 3-fold induction of CCA1 expression at noon (FIG. 4a) and 1.5-30-fold repression of the downstream genes PORA, PORB, AMY, DPE1, and GWD (FIG. 10a), resulting in ˜14% and ˜17% reduction of chlorophyll and starch contents, respectively (FIG. 4a). CCA1-OX had ˜20% reduction of chlorophyll content in seedlings (FIG. 9c) and may affect various regulators in clock and other pathways related to growth vigor. For example, gi mutants in A. thaliana increase starch content and flower late, but GI induction in the allotetraploids correlates with starch accumulation. CCA1-OX lines also flowered late and may increase chlorophyll and starch content in late stages.

To test whether CCA1 repression has positive effects on growth vigor in diploids as in the hybrids and allotetraploids (FIG. 2a and FIG. 3, a-e), starch content in cca1 single and cca1 lhy double mutants was examined. CCA1 expression was not completely abolished in these mutants (FIG. 4b) probably because of the T-DNA insertion near the ATG codon. The five downstream genes examined were upregulated 1.5-12.5-fold in the mutants (FIG. 10b), and the starch content was doubled in the cca1 mutant (FIG. 4b). The starch content was lower in the double mutant than in cca1, indicating a metabolic penalty of severely lacking clock regulation. Furthermore, to reduce CCA1 expression during the day, we expressed cca1-RNAi driven by the TOC1 promoter (FIG. 10c). In the TOC1:cca1-RNAi transgenic plants, CCA1 mRNA and protein levels were down-regulated 2-10 fold (FIG. 4c, left) and 1.4-3 fold (right), respectively. Consequently, four downstream genes examined were upregulated in the TOC1:cca1-RNAi lines (FIG. 10e), and the starch content increased ˜28% (FIG. 4d). Taken together, the data suggest a mechanistic role of CCA1 repression in promoting downstream pathways, increasing chlorophyll and starch accumulation and growth vigor.

A model is proposed that explains growth vigor and increased biomass in allotetraploids and hybrids (FIG. 4e). Correct circadian regulation enhances fitness and metabolism. In the allotetraploids the expression of clock regulators is altered through autonomous regulation and chromatin modifications (FIG. 1d), including rhythmic changes in H3 acetylation in the TOC1 promoter. During the day, A. thaliana CCA1 (and LHY) is epigenetically repressed, leading to upregulation of EE- and CBS-containing downstream genes in photosynthesis and carbohydrate metabolism. As a result, the entire network is reset at a high amplitude during the day, increasing chlorophyll synthesis and starch metabolism. At night CCA1 is derepressed and resumes normal oscillation. Although little is known about why the A. thaliana genes are repressed during the day, the repression is likely associated with cis- and trans-acting effects on homoeologous loci in the allotetraploids, as observed in flowering-time genes.

Interestingly, modulation of circadian clock regulators in allopolyploids and hybrids is reminiscent of switching gene expression during dawn- and evening-phased rhythmic alternation that is required for properly maintaining homoeostasis in clock-mediated metabolic pathways in diploids. Hybrids and allopolyploids simply exploit epigenetic modulation of parental alleles and homoeologous loci of the internal clock regulators and use this convenient mechanism to alter the amplitude of gene expression and metabolic flux and gain advantages from clock-mediated photosynthesis and carbohydrate metabolism.

Epigenetic regulation of a few regulatory genes induces cascade changes in downstream genes and physiological pathways and ultimately growth and development, which provides a general mechanism for growth vigor and increased biomass that are commonly observed in the hybrids and allopolyploids produced within and between species.

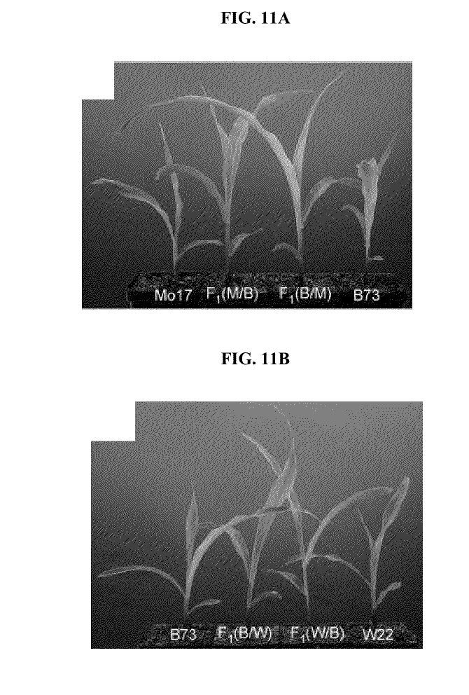

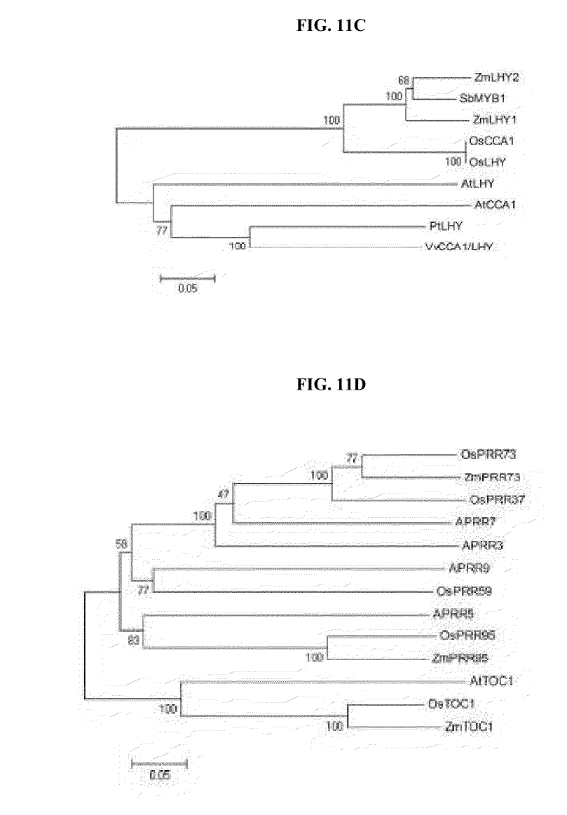

Growth vigor was also found in the seedlings of the reciprocal hybrids of two pairs of maize inbred lines, namely Mo17 and B73 (FIG. 11a) and B73 and W22 (FIG. 11b). The F₁ seedlings were 10-15% taller and larger than the parents, although they had similar developmental stages.

FIG. 11c displayed high conservation of circadian clock genes in Arabidopsis, poplar, grapevine, rice, sorghum, and maize. CCA1 genes are grouped in two clades, a clade for dicots (Arabidopsis, poplar, and grapevine) and a Glade for monocots (rice, sorghum, and maize). Amino acid sequences of A. thaliana CCA1 is most closely related to that of poplar and grapevine. Rice has both CCA1 and LHY, whereas maize contains two LHY homologs but no obvious CCA1 homolog. Only CCA1 homolog found in sorghum is a predicted MYB1 protein. The genes in monocots more closely related in maize and The data suggest genetic variation of CCA1 and LHY genes, which may contribute to different growth patterns in these plant species.

TOC1 homologs were conserved in Arabidopsis, rice, and maize (FIG. 11d). In addition, several clock-associated pseudo-response regulator (APRR) homologs were identified in rice and maize. Conservation of CCA1, LHY, and TOC1 genes suggests that a similar molecular clock controls growth vigor in hybrids of maize and rice. Down-regulation of CCA1-like gene was also found after the analysis of public microarray data performed in F₁ hybrids of Mo17 and B73.

Genetic mapping studies indicated that many life history traits including plant height and leaf length and number were coincidentally mapped in the locations of CCA1 (bottom of chromosome 2) and LHY (top of chromosome 1) in the recombinant inbred lines (RILs) derived from Ler and Cvi. Another locus Cryptochrome 2 (CRY2) in the vicinity of LHY was also a candidate for fruit length and ovule number but not for other traits. CRY2 is blue light photoreceptor and is involved in circadian clock regulation in plants and animals.

Mammalian CRY1 and CRY2 have co-opted the role in the maintenance of circadian rhythms and are essential components of the negative limb of the circadian clock feedback loop. This suggests that circadian clocks and their associated regulation for physiology and metabolism are conserved across plant and animal kingdom.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Table 6 summarizes the locations of CCA1 binding site (CBS) or evening element (EE) in the downstream genes.

TABLE 6

| Locus | Name | CBS/EE | Position |
|---|---|---|---|
| At5g26570 | GWD3 | AAAAaATCT | −28 −19 |
| At1g69830 | AMY3 | AAAATATCT | −47 −38 |
| At5g64860 | DPEl | AAAATATCT | −80 −71 |
| At3g29320 | PHSl | gAAATATCT | −218 −209 |
| At5g54190 | PORA | AAAATATCT | −222 −213 |
| At3g46970.1 | PHS2 | AAAATATCT | −235 −226 |
| At4g27440 | PORB | AAAATATCT | −245 −236 |
| At2g40840 | DPE2 | AAAATATCT | −330 −321 |
| At1g10760 | GWDl | AAAATATCT | −295 −286 |
| At2g39930 | ISAl | AAAAAATCT | −400 −391 |
| At5g61380.1 | Toel | AAAATATCT | −40 −31 |
| At1g22770 | GI | AAAATATCT | −729 −720 |
| | | AAAATATCT | −1239 −1230 |
| | | AAAATATCT | −1457 −1448 |

Table 7 lists the 128 upregulated genes and CBS or EE motif locations.

TABLE 7

| | | Allo738/MPV | | Allo733/MPV | | (AA)AATATCT | | | | | | |
|---|---|---|---|---|---|---|---|---|---|---|---|---|
| GeneID | AGI | FoldChang | Pvalue | FoldChang | Pvalue | 2000- | 1500 | [symbol] 500- | 100 [symbol] | 1000- | 500 | 500- |
| A020045_( | At3g2674 | 4.8191 | 0.000151 | 2.2435 | 4.03E−06 | | | 1 | | | 1 | |
| A002062_( | At1g6983 | 4.3907 | 5.37E−09 | 2.7331 | 4.18E−06 | | 1 | | | | | |
| A019512_( | At4g3234 | 3.7485 | 7.18E−08 | 1.798 | 2.72E−05 | | | | | | | |
| A001518_( | At5g4962 | 2.4049 | 1.09E−07 | 3.8606 | 4.45E−07 | | | | | 1 | | |
| A017467_( | At5g6235 | 2.404 | 2.63E−06 | 1.9525 | 4.39E−05 | | | 1 | | | | |
| A018600_( | At5g6486 | 2.2504 | 6.98E−07 | 1.6357 | 1.24E−06 | | | | | | | |
| A017042_( | At1g2160 | 2.2193 | 0.000153 | 1.6336 | 0.002358 | | | | | | | 1 |
| A002820_( | At2g4358 | 2.1291 | 1.97E−07 | 1.7338 | 1.83E−05 | | | | | | | |
| A023336_( | At2g1597 | 1.9484 | 0.000375 | 1.8988 | 0.000116 | | | | | | | |
| A008602_( | At4g2744 | 1.8498 | 9.14E−09 | 1.8603 | 8.68E−07 | | | | | | | |
| A021034_( | At4g2744 | 8.9261 | 1.63E−07 | 7.7572 | 2.95E−08 | | | | | | 1 | |
| A020917_( | At1g7538 | 3.8137 | 3.26E−06 | 1.6821 | 1.41E−05 | | | | | | 1 | |
| A003431_( | At1g0646 | 2.9076 | 4.19E−09 | 1.6105 | 5.93E−06 | | | | | | | |
| A021216_( | At4g3371 | 2.6569 | 1.16E−06 | 1.9102 | 7.79E−07 | 1 | | | | | | |
| A016420_( | At5g0216 | 2.2176 | 4.30E−06 | 1.7852 | 2.30E−06 | | 1 | | | | | |
| A024454_( | At3g2212 | 2.1741 | 3.16E−06 | 2.4629 | 3.77E−07 | | | | | | 1 | |
| A010449_( | At3g0892 | 2.126 | 9.05E−07 | 1.6756 | 4.50E−07 | | | | | | | |
| A001481_( | At5g2063 | 2.0749 | 3.05E−07 | 1.6962 | 1.53E−05 | | | | | | | |
| A018891_( | At5g1014 | 1.8013 | 1.67E−07 | 2.3243 | 3.49E−07 | | | | | 1 | | |
| A019105_( | At2g2284 | 1.7504 | 0.000264 | 2.1992 | 3.69E−05 | | | | 2 | | | |
| A023455_( | At3g4697 | 3.0725 | 2.36E−05 | 1.7882 | 0.004138 | 1 | 1 | | | | | |
| A019485_( | At2g4084 | 4.4848 | 1.93E−06 | 2.8814 | 5.91E−05 | | | | | | | |
| A006869_( | At5g5419 | 2.431 | 1.25E−06 | 2.208 | 3.56E−05 | | | | | | | |
| A005183_( | At1g1327 | 2.2022 | 7.97E−07 | 2.6327 | 6.73E−05 | | | | | | 1 | |
| A000005_( | At2g2138 | 2.1468 | 0.012295 | 2.0541 | 0.000337 | | | | | | | |
| A005372_( | At3g1317 | 1.9469 | 0.00011 | 1.7611 | 0.000101 | | | | | | | |
| A012515_( | At2g2963 | 1.8872 | 1.47E−07 | 1.8678 | 0.001005 | 1 | | | | | | |
| A008658_( | At5g5551 | 2.9549 | 2.03E−07 | 1.9195 | 2.13E−05 | | | | 1 | | 3 | 1 |
| A018614_( | At1g1020 | 2.0936 | 7.71E−06 | 1.6958 | 0.00725 | | | | | | | 1 |
| A001933_( | At1g1076 | 1.9226 | 1.93E−06 | 1.9967 | 7.73E−06 | | | | | | | 1 |
| A001393_( | At5g2561 | 6.9786 | 1.13E−06 | 3.0602 | 2.96E−06 | | | 1 | | 2 | 1 | |
| A005592_( | At4g3066 | 1.7286 | 1.67E−05 | 1.9633 | 0.000191 | | | | | 2 | 1 | |
| A013749_( | At3g1557 | 3.4981 | 4.38E−07 | 2.4314 | 7.26E−05 | | | | | 1 | 1 | |
| A019627_( | At4g2500 | 3.3382 | 4.69E−06 | 3.5005 | 8.19E−07 | | | | 1 | 1 | | |
| A014046_( | At1g2007 | 2.8163 | 1.13E−07 | 1.6763 | 0.000384 | | | | | 1 | | |
| A000325_( | At1g6243 | 2.7732 | 3.63E−06 | 1.9846 | 3.09E−05 | | | | | 1 | 2 | |
| A021054_( | At1g3025 | 2.0421 | 5.34E−05 | 1.9772 | 0.001427 | | | | | 1 | 1 | |
| A000993_( | At3g4786 | 2.0093 | 0.000173 | 1.8665 | 8.02E−06 | | | | | 1 | | |
| A009934_( | At3g4786 | 1.9615 | 8.42E−05 | 2.3474 | 3.33E−08 | | | | | 1 | | |
| A009127_( | At3g1293 | 1.9546 | 2.24E−05 | 1.8098 | 1.98E−06 | | | | | 1 | | |
| A006288_( | At2g0342 | 1.9278 | 1.31E−06 | 1.6131 | 3.12E−05 | | 2 | | | 1 | | |
| A020883_( | At2g3993 | 1.7549 | 1.78E−05 | 1.6345 | 7.49E−07 | | | | | 1 | | |
| A009434_( | At3g5772 | 1.7261 | 3.95E−05 | 2.0559 | 5.47E−07 | | 1 | | | 1 | | |
| A024703_( | At4g0331 | 1.7023 | 1.65E−06 | 2.3405 | 8.96E−07 | | | | | 1 | | |
| A019449_( | At1g2277 | 3.3552 | 3.95E−06 | 1.7606 | 8.51E−06 | | | 2 | | | 1 | |
| A020363_( | At2g3980 | 2.8471 | 2.96E−07 | 2.0604 | 2.42E−05 | | | 1 | | | | |
| A020233_( | At4g3747 | 2.8296 | 1.96E−07 | 1.7086 | 0.000108 | | | 1 | | | | |
| A017657_( | At5g4593 | 2.413 | 7.52E−08 | 1.9732 | 1.20E−06 | | 1 | 1 | | | | |
| A025734_( | At1g6069 | 2.3683 | 0.000308 | 1.7841 | 7.78E−05 | | | 1 | | | | |
| A001363_( | At1g2382 | 1.7879 | 2.86E−06 | 1.6178 | 0.000742 | | | 1 | | | | |
| A002862_( | At1g0939 | 1.7021 | 5.52E−05 | 1.8988 | 4.69E−05 | | | 1 | 1 | | | |
| A003656_( | At1g7497 | 1.2634 | 7.11E−15 | 1.5655 | 0 | | 1 | 1 | | | | |
| A011016_( | At3g2813 | 1.7474 | 4.47E−06 | 1.7957 | 0.000101 | 3 | 1 | | | | 1 | |
| A014515_( | At4g0433 | 4.1537 | 1.03E−07 | 1.8602 | 1.82E−05 | 1 | 1 | | 1 | | | |
| A002709_( | At1g2003 | 3.5455 | 4.92E−06 | 1.7235 | 0.000971 | 1 | | | | | | |
| A012985_( | At4f1916 | 3.5432 | 1.40E−07 | 1.6483 | 0.000317 | 1 | | | | | | |
| A023853_( | At1g4737 | 3.2937 | 7.10E−07 | 2.4645 | 4.41E−05 | 1 | | | 1 | | | |
| A017543_( | At5g1158 | 2.6992 | 6.31E−06 | 1.6651 | 0.000419 | 1 | | | | | | |
| A015908_( | At5g6378 | 2.6239 | 1.01E−05 | 2.0363 | 7.39E−05 | 1 | 1 | | | | | |
| A010658_( | At3g5561 | 2.2028 | 8.31E−07 | 1.9422 | 5.89 | 1 | | | | 1 | | |
| A009505_( | At3g2162 | 2.0357 | 5.62E−07 | 1.6179 | 5.89 | 1 | | | | | | |
| A007555_( | At2g4464 | 1.8276 | 1.97E−07 | 1.721 | 0.000259 | 1 | | | | 1 | | |
| A022609_( | At4g1548 | 1.7597 | 1.51E−05 | 1.7458 | 2.75 | 1 | | | | | | |
| A011614_( | At3g1424 | 1.7398 | 1.95E−07 | 1.6961 | 2.00E−05 | 1 | | | 1 | 1 | | |
| A012773_( | At3g1808 | 6.2657 | 8.23E−08 | 2.7853 | 2.44E−06 | | | | | | | |
| A008731_( | At2g2890 | 1.9941 | 1.67E−06 | 1.7787 | 2.08E−06 | | | | | | | |
| A021081_( | At3g5346 | 2.7516 | 9.19E−06 | 2.0829 | 2.45E−05 | | | | | | | |
| A019523_( | At4g3498 | 1.8761 | 3.92E−07 | 1.7551 | 7.14E−06 | | | | | | | |
| A010673_( | At3g4798 | 2.0811 | 1.39E−05 | 1.7384 | 5.83E−06 | | | | | 1 | | |
| A006796_( | At2g4359 | 2.4577 | 2.48E−06 | 2.1806 | 9.75E−07 | | | | | 1 | | |
| A017118_( | At5g6166 | 1.7442 | 3.05E−07 | 1.7584 | 9.23E−06 | | | 1 | | 2 | | |
| A020107_( | At5g4383 | 2.2163 | 1.59E−05 | 1.6579 | 2.18E−05 | | | | | 1 | | |
| A001263_( | At1g2038 | 2.1958 | 1.15E−05 | 1.8114 | 4.27E−15 | | | | | 1 | | |
| A022887_( | At5g2444 | 2.1933 | 2.22E−05 | 1.8946 | 1.96E−06 | | 2 | | | 1 | | |
| A014704_( | At4g3926 | 3.2567 | 4.60E−05 | 2.0674 | 3.15E−06 | | | 1 | 1 | | | |
| A021522_( | At1g6206 | 2.821 | 6.09E−09 | 2.7332 | 8.85E−09 | | | | 1 | | | |
| A006636_( | At2g1482 | 2.7388 | 9.02E−06 | 1.8637 | 1.82E−06 | | | | 1 | | | |
| A010415_( | At3g4675 | 2.5411 | 2.60E−05 | 1.7577 | 0.000435 | | | | 1 | | | |
| A015071_( | At4g2510 | 2.4968 | 4.37E−06 | 2.5257 | 0.001612 | | | 1 | 1 | | | |
| A001387_( | At1g0130 | 2.3739 | 5.79E−06 | 2.595 | 2.88E−05 | | | | 1 | | | |
| A010894_( | At3g1229 | 2.219 | 2.15E−05 | 2.0208 | 3.68E−06 | | | | 1 | | | |
| A009216_( | At3g2297 | 2.1265 | 8.71E−06 | 1.8682 | 0.000192 | | | | 1 | | | |
| A001231_( | At1g6394 | 1.9757 | 3.03E−06 | 2.0434 | 1.47E−07 | | | | 1 | | | |
| A021731_( | At3g1243 | 1.9373 | 0.000192 | 1.6457 | 0.005896 | | | 1 | | | | |
| A005991_( | At5g4412 | 1.9311 | 6.29E−08 | 1.7641 | 1.39E−06 | | | 1 | | | | |
| A019461_( | At3g5050 | 3.3402 | 7.55E−08 | 1.894 | 4.48E−07 | | 1 | | | | | |
| A025241_( | At5g1693 | 3.105 | 9.66E−07 | 1.929 | 0.000284 | | 1 | | | | | |
| A015099_( | At4g0902 | 2.9855 | 1.28E−05 | 1.714 | 0.015731 | | 1 | | | | | |
| A013821_( | At4g3212 | 2.4123 | 1.37E−07 | 1.8172 | 7.80E−05 | | 1 | | | | | |
| A023088_( | At5g5181 | 2.2152 | 6.75E−06 | 1.6601 | 0.000194 | | 1 | | | | | |
| A010858_( | At3g2202 | 1.8938 | 0.000292 | 1.6227 | 0.000998 | | 1 | | | | | |
| A001251_( | At1g1083 | 1.7222 | 4.67E−05 | 1.6799 | 0.000225 | | 1 | | | | | |

| | (AA)AATATCT | | | | | | | | | |
|---|---|---|---|---|---|---|---|---|---|---|
| GeneID | 400 | 400- | 300 | 300- | 200 | 200- | 100 | 100- | ATG | description |
| A020045_( | | | | 1 | | | 1 | 1 | | ‘light regulated protein-related |
| A002062_( | | | 1 | | | | | 1 | | alpha-amylase (1.4-alpha-D-glucan |
| A019512_( | | | | | | | | 1 | | ‘expressed protein |
| A001518_( | | | | | | | | 1 | | ‘expressed protein |
| A017467_( | | | | | | | | 1 | | ‘myb family transcription factor |
| A018600_( | | | | | | | | 1 | | ‘pectinesterase family |
| A017042_( | | | | | | | | 1 | | ‘glycosyl hydrolase family 77 (4-alpha- |
| A002820_( | | | | | | | | | | ‘expressed protein |
| A023336_( | | | | | | | | | | ‘glycosly hydrolase family 19 (chitinase) |
| A008602_( | | | | | | 2 | | | | ‘cold acclimation protein WCOR413 (Triticum |
| A021034_( | | | | 1 | | 1 | | | 1 | ‘protochlorophyllide reductase B (PCR B/POR |
| A020917_( | | | | | | 1 | | | | wound-responsive protein-related |
| A003431_( | | | | | | 1 | | | | ‘heat shock protein family |
| A021216_( | | | | | | 1 | | | | ‘pathogenesis-related protein, putative |
| A016420_( | | | | | | 1 | | | | ‘express protein |
| A024454_( | | | | | | 1 | | | | ‘protease inhibitor/seed storag/lipid transfer |
| A010449_( | | | | | | 1 | | | 1 | ‘rhodanese-like domain protein |
| A001481_( | | | | | | 1 | | | | ATP-dependent protease La (ON) domain- |
| A018891_( | | | | | | 1 | | | | ‘germin-like protein (AtGER3) |
| A019105_( | | | | | | 1 | | | | ‘MADS box protein FLOWERING LOCUS F |
| A023455_( | | | | | | 1 | | | | ‘transcription activator (GRL1) |
| A019485_( | | | | 1 | | | | | | ‘starch phosphorylase, putative |
| A006869_( | | | | 1 | | | | | | ‘glycosyl hydrolase family 77 (4-alpha- |
| A005183_( | | | | 1 | | | | | | ‘protochlorophyllide reductase A (PCR A/POR |
| A000005_( | | 1 | | 1 | | | | | 1 | ‘methionine aminopeptidase I (MAP1)-related |
| A005372_( | | 1 | | | | | | | | ‘kinesin-related protein |
| A012515_( | | | | | | | | | | express protein |
| A008658_( | | | | | | | | | | ‘thiamin biosynthesis protein-related |
| A018614_( | | | | | | | | | | expressed protein/similar to mitochondrial impo [symbol] |
| A001933_( | | | | | | | | | | transcription factor-related/strong similarity to |
| A001393_( | | | | | | | | | | ‘SEX1 protein; nuclear gene for chloroplast |
| A005592_( | | | | | | | | | | ‘dehydration-induced protein RD22 |
| A013749_( | | | | | | | 1 | | | ‘stress responsive protein homolog |
| A019627_( | | | | | | | | | | ‘non-phototropic hypocotyl protein-related |
| A014046_( | | | | | 1 | | | | | ‘alpha-amylase (1,4-alpha-D-glucan |
| A000325_( | | | | | | | | | | ‘expressed protein |
| A021054_( | | | | | | | | | | ‘phosphatidate cytidylyltransfe (CDS) |
| A000993_( | 1 | | | | | | | | | express protein |
| A009934_( | | | | | 1 | | | | | express protein |
| A009127_( | | | | | | | 1 | | | express protein |
| A006288_( | | | | | | | | | | express protein |
| A020883_( | | | | | | | | | | ‘isoamylase, putative |
| A009434_( | | | | | | | | | | ‘protein kinase, putative |
| A024703_( | | | | | | | | | | ‘plant transposase (Ptta/En/Spm) family |
| A019449_( | 1 | | 1 | | | | | | | ‘gigantea protein-related |
| A020363_( | | | | | | | | | | ‘delta 1-pyrroline-5-carboxylate synthetase A |
| A020233_( | | | | | | | | | 1 | ‘hydrolase, alpha/beta fold family |
| A017657_( | | | | | | | | | | ‘magnesium-chelatase, subunit chil, chloroplast |
| A025734_( | | | | | | | | | | ‘aldo/keto reductase family |
| A001363_( | | | | | | | | | | spermidine synthase-related |
| A002862_( | | | | | | | | | | GDSL-motif lipase/hydrolase protein |
| A003656_( | | | | | | | | | | ribosomal protein S9-related |
| A011016_( | | | | | | | | | | nodulin MtN21 family protein |
| A014515_( | | | | | | | | | | expressed protein |
| A002709_( | 2 | | | | | | | | | thaumatin family |
| A012985_( | | | | | | | | | | expressed protein |
| A023853_( | | | | | | | | | | disease resistance protein (TIR class), putative |
| A017543_( | | | | | | | | | | expressed protein/UVB-resistance protein-relate [symbol] |
| A015908_( | | | | | | | | | | ‘zinc finger (c3Hc4-type RING finger) protein |
| A010658_( | | | | | | | | | | delta 1-pyrroline-5-carboxylate synthetase B (P [symbol] |
| A009505_( | | | | | | | | | | ‘ERD4 protein-related |
| A007555_( | | | | | | | | | | expressed protein |
| A022609_( | | | | | | | | | 1 | ‘UDP = glycosyltransferase family |
| A011614_( | | | | | | | | | | subtilisin-like serine protease |
| A012773_( | | | | | | | | | 1 | unknown |
| A008731_( | | | | | | | 1 | | 1 | membrane channel protein-related |
| A021081_( | | | | | | | 1 | | | 29 kDa ribonucleoprotein, chloroplast (RNA-bin [symbol] |
| A019523_( | 1 | | 1 | | | | 1 | | | subtilisin-like serine protease |
| A010673_( | | | | | 2 | | | | | expressed protein |
| A006796_( | | | | | 1 | | | | | glycosyl hydrolase family 19 (chitinase) |
| A017118_( | | | | | | | | | | glycine-rich protein |
| A020107_( | | | | | | | | | | ‘aluminium-induced protein-related |
| A001263_( | | | | | | | | | | prolyl endopeptidase-related |
| A022887_( | | | | | | | | | | RNA-binding protein, putative |
| A014704_( | | | | | | | | | | glycine-rich RNA-binding protein 8 (GRP8) (CCI |
| A021522_( | | | | | | | | | | expressed protein |
| A006636_( | | | | | | | | | | photoropic response protein family |
| A010415_( | | | | | | | | | | hypothetical protein |
| A015071_( | | | | | | | | | | iron superoxide dismutase (FSD1) |
| A001387_( | | | | | | | | | | chloroplast nucleoid DNA binding protein-relate [symbol] |
| A010894_( | | | | | | | | | | tetrahydrofolate dehydrogenase/cyclohydrolase |
| A009216_( | | | | | | | | | | expressed protein |
| A001231_( | | | | | | | | | | monodehydroascorbate reductase, putative |
| A021731_( | | | | | | | | | | hypothetical protein |
| A005991_( | | | | | | | | | | 12S seed storage protein (CRA1) |
| A019461_( | | | | | | | | | | protein kinase, putative |
| A025241_( | | | | | | | | | | expressed protein |
| A015099_( | | | | | | | | | | glycoside hydrolase family 13 |
| A013821_( | | | | | | | | | | galactosyltransferase family |
| A023088_( | | | | | | | | | | gibberellin 20-oxidase, putative |
| A010858_( | | | | | | | | | | hypothetical protein |
| A001251_( | | | | | | | | | | hypothetical protein |

Table 7A

Allo738/MPV

| GeneID | AGI | FoldChang | Pvalue |
|---|---|---|---|
| A020045_( | At3g2674 | 4.8191 | 0.000151 |
| A002062_( | At1g6983 | 4.3907 | 5.37E−09 |
| A019512_( | At4g3234 | 3.7485 | 7.18E−08 |
| A001518_( | At5g4962 | 2.4049 | 1.09E−07 |
| A017467_( | At5g6235 | 2.404 | 2.63E−06 |
| A018600_( | At5g6486 | 2.2504 | 6.98E−07 |
| A017042_( | At1g2160 | 2.2193 | 0.000153 |
| A002820_( | At2g4358 | 2.1291 | 1.97E−07 |
| A023336_( | At2g1597 | 1.9484 | 0.000375 |
| A008602_( | At4g2744 | 1.8498 | 9.14E−09 |
| A021034_( | At4g2744 | 8.9261 | 1.63E−07 |
| A020917_( | At1g7538 | 3.8137 | 3.26E−06 |
| A003431_( | At1g0646 | 2.9076 | 4.19E−09 |
| A021216_( | At4g3371 | 2.6569 | 1.16E−06 |
| A016420_( | At5g0216 | 2.2176 | 4.30E−06 |
| A024454_( | At3g2212 | 2.1741 | 3.16E−06 |
| A010449_( | At3g0892 | 2.126 | 9.05E−07 |
| A001481_( | At5g2063 | 2.0749 | 3.05E−07 |
| A018891_( | At5g1014 | 1.8013 | 1.67E−07 |
| A019105_( | At2g2284 | 1.7504 | 0.000264 |
| A023455_( | At3g4697 | 3.0725 | 2.36E−05 |
| A019485_( | At2g4084 | 4.4848 | 1.93E−06 |
| A006869_( | At5g5419 | 2.431 | 1.25E−06 |
| A005183_( | At1g1327 | 2.2022 | 7.97E−07 |
| A000005_( | At2g2138 | 2.1468 | 0.012295 |
| A005372_( | At3g1317 | 1.9469 | 0.00011 |
| A012515_( | At2g2963 | 1.8872 | 1.47E−07 |
| A008658_( | At5g5551 | 2.9549 | 2.03E−07 |
| A018614_( | At1g1020 | 2.0936 | 7.71E−06 |
| A001933_( | At1g1076 | 1.9226 | 1.93E−06 |
| A001393_( | At5g2561 | 6.9786 | 1.13E−06 |
| A005592_( | At4g3066 | 1.7286 | 1.67E−05 |
| A013749_( | At3g1557 | 3.4981 | 4.38E−07 |
| A019627_( | At4g2500 | 3.3382 | 4.69E−06 |
| A014046_( | At1g2007 | 2.8163 | 1.13E−07 |
| A000325_( | At1g6243 | 2.7732 | 3.63E−06 |
| A021054_( | At1g3025 | 2.0421 | 5.34E−05 |
| A000993_( | At3g4786 | 2.0093 | 0.000173 |
| A009934_( | At3g4786 | 1.9615 | 8.42E−05 |
| A009127_( | At3g1293 | 1.9546 | 2.24E−05 |
| A006288_( | At2g0342 | 1.9278 | 1.31E−06 |
| A020883_( | At2g3993 | 1.7549 | 1.78E−05 |
| A009434_( | At3g5772 | 1.7261 | 3.95E−05 |
| A024703_( | At4g0331 | 1.7023 | 1.65E−06 |
| A019449_( | At1g2277 | 3.3552 | 3.95E−06 |
| A020363_( | At2g3980 | 2.8471 | 2.96E−07 |
| A020233_( | At4g3747 | 2.8296 | 1.96E−07 |
| A017657_( | At5g4593 | 2.413 | 7.52E−08 |
| A025734_( | At1g6069 | 2.3683 | 0.000308 |
| A001363_( | At1g2382 | 1.7879 | 2.86E−06 |
| A002862_( | At1g0939 | 1.7021 | 5.52E−05 |
| A003656_( | At1g7497 | 1.2634 | 7.11E−15 |
| A011016_( | At3g2813 | 1.7474 | 4.47E−06 |
| A014515_( | At4g0433 | 4.1537 | 1.03E−07 |
| A002709_( | At1g2003 | 3.5455 | 4.92E−06 |
| A012985_( | At4f1916 | 3.5432 | 1.40E−07 |
| A023853_( | At1g4737 | 3.2937 | 7.10E−07 |
| A017543_( | At5g1158 | 2.6992 | 6.31E−06 |
| A015908_( | At5g6378 | 2.6239 | 1.01E−05 |
| A010658_( | At3g5561 | 2.2028 | 8.31E−07 |
| A009505_( | At3g2162 | 2.0357 | 5.62E−07 |
| A007555_( | At2g4464 | 1.8276 | 1.97E−07 |
| A022609_( | At4g1548 | 1.7597 | 1.51E−05 |
| A011614_( | At3g1424 | 1.7398 | 1.95E−07 |
| A012773_( | At3g1808 | 6.2657 | 8.23E−08 |
| A008731_( | At2g2890 | 1.9941 | 1.67E−06 |
| A021081_( | At3g5346 | 2.7516 | 9.19E−06 |
| A019523_( | At4g3498 | 1.8761 | 3.92E−07 |
| A010673_( | At3g4798 | 2.0811 | 1.39E−05 |
| A006796_( | At2g4359 | 2.4577 | 2.48E−06 |
| A017118_( | At5g6166 | 1.7442 | 3.05E−07 |
| A020107_( | At5g4383 | 2.2163 | 1.59E−05 |
| A001263_( | At1g2038 | 2.1958 | 1.15E−05 |
| A022887_( | At5g2444 | 2.1933 | 2.22E−05 |
| A014704_( | At4g3926 | 3.2567 | 4.60E−05 |
| A021522_( | At1g6206 | 2.821 | 6.09E−09 |
| A006636_( | At2g1482 | 2.7388 | 9.02E−06 |
| A010415_( | At3g4675 | 2.5411 | 2.60E−05 |
| A015071_( | At4g2510 | 2.4968 | 4.37E−06 |
| A001387_( | At1g0130 | 2.3739 | 5.79E−06 |
| A010894_( | At3g1229 | 2.219 | 2.15E−05 |
| A009216_( | At3g2297 | 2.1265 | 8.71E−06 |
| A001231_( | At1g6394 | 1.9757 | 3.03E−06 |
| A021731_( | At3g1243 | 1.9373 | 0.000192 |
| A005991_( | At5g4412 | 1.9311 | 6.29E−08 |
| A019461_( | At3g5050 | 3.3402 | 7.55E−08 |
| A025241_( | At5g1693 | 3.105 | 9.66E−07 |
| A015099_( | At4g0902 | 2.9855 | 1.28E−05 |
| A013821_( | At4g3212 | 2.4123 | 1.37E−07 |
| A023088_( | At5g5181 | 2.2152 | 6.75E−06 |
| A010858_( | At3g2202 | 1.8938 | 0.000292 |
| A001251_( | At1g1083 | 1.7222 | 4.67E−05 |

Table 7B

Allo733/MPV

| GeneID | Fold Chang | Pvalue | description |
|---|---|---|---|
| A020045_( | 2.2435 | 4.03E−06 | ‘light regulated protein-related |
| A002062_( | 2.7331 | 4.18E−06 | alpha-amylase (1.4-alpha-D-g |
| A019512_( | 1.798 | 2.72E−05 | ‘expressed protein |
| A001518_( | 3.8606 | 4.45E−07 | ‘expressed protein |
| A017467_( | 1.9525 | 4.39E−05 | ‘myb family transcription factor |
| A018600_( | 1.6357 | 1.24E−06 | ‘pectinesterase family |
| A017042_( | 1.6336 | 0.002358 | ‘glycosyl hydrolase family 77 ( |
| A002820_( | 1.7338 | 1.83E−05 | ‘expressed protein |
| A023336_( | 1.8988 | 0.000116 | ‘glycosly hydrolase family 19 ( |
| A008602_( | 1.8603 | 8.68E−07 | ‘cold acclimation protein WCO |
| A021034_( | 7.7572 | 2.95E−08 | ‘protochlorophyllide reductase |
| A020917_( | 1.6821 | 1.41E−05 | wound-responsive protein-rela |
| A003431_( | 1.6105 | 5.93E−06 | ‘heat shock protein family |
| A021216_( | 1.9102 | 7.79E−07 | ‘pathogenesis-related protein, |
| A016420_( | 1.7852 | 2.30E−06 | ‘express protein |
| A024454_( | 2.4629 | 3.77E−07 | ‘protease inhibitor/seed storag |
| A010449_( | 1.6756 | 4.50E−07 | ‘rhodanese-like domain protein |
| A001481_( | 1.6962 | 1.53E−05 | ATP-dependent protease La ( |
| A018891_( | 2.3243 | 3.49E−07 | ‘germin-like protein (AtGER3) |
| A019105_( | 2.1992 | 3.69E−05 | ‘MADS box protein FLOWERIN |
| A023455_( | 1.7882 | 0.004138 | ‘transcription activator (GRL1) |
| A019485_( | 2.8814 | 5.91E−05 | ‘starch phosphorylase, putative |
| A006869_( | 2.208 | 3.56E−05 | ‘glycosyl hydrolase family 77 ( |
| A005183_( | 2.6327 | 6.73E−05 | ‘protochlorophyllide reductase |
| A000005_( | 2.0541 | 0.000337 | ‘methionine aminopeptidase I |
| A005372_( | 1.7611 | 0.000101 | ‘kinesin-related protein |
| A012515_( | 1.8678 | 0.001005 | express protein |
| A008658_( | 1.9195 | 2.13E−05 | ‘thiamin biosynthesis protein |
| A018614_( | 1.6958 | 0.00725 | expressed protein/similar to m |
| A001933_( | 1.9967 | 7.73E−06 | transcription factor-related.stn |
| A001393_( | 3.0602 | 2.96E−06 | ‘SEX1 protein; nuclear gene fo |
| A005592_( | 1.9633 | 0.000191 | ‘dehydration-induced protein R |
| A013749_( | 2.4314 | 7.26E−05 | ‘stress responsive protein hom |
| A019627_( | 3.5005 | 8.19E−07 | ‘non-phototropic hypocotyl pro |
| A014046_( | 1.6763 | 0.000384 | ‘alpha-amylase (1,4-alpha-D-g |
| A000325_( | 1.9846 | 3.09E−05 | ‘expressed protein |
| A021054_( | 1.9772 | 0.001427 | ‘phosphatidate cytidylyltransfe |
| A000993_( | 1.8665 | 8.02E−06 | express protein |
| A009934_( | 2.3474 | 3.33E−08 | express protein |
| A009127_( | 1.8098 | 1.98E−06 | express protein |
| A006288_( | 1.6131 | 3.12E−05 | express protein |
| A020883_( | 1.6345 | 7.49E−07 | ‘isoamylase, putative |
| A009434_( | 2.0559 | 5.47E−07 | ‘protein kinase, putative |
| A024703_( | 2.3405 | 8.96E−07 | ‘plant transposase (Ptta/En/Sp |
| A019449_( | 1.7606 | 8.51E−06 | ‘gigantea protein-related |
| A020363_( | 2.0604 | 2.42E−05 | ‘delta 1-pyrroline-5-carboxylate |
| A020233_( | 1.7086 | 0.000108 | ‘hydrolase, alpha/beta fold fam |
| A017657_( | 1.9732 | 1.20E−06 | ‘magnesium-chelatase, subun |
| A025734_( | 1.7841 | 7.78E−05 | ‘aldo/keto reductase family |
| A001363_( | 1.6178 | 0.000742 | spermidine synthase-related |
| A002862_( | 1.8988 | 4.69E−05 | GDSL-motif lipase/hydrolase p |
| A003656_( | 1.5655 | 0 | ribosomal protein S9-related |
| A011016_( | 1.7957 | 0.000101 | nodulin MtN21 family protein |
| A014515_( | 1.8602 | 1.82E−05 | expressed protein |
| A002709_( | 1.7235 | 0.000971 | thaumatin family |
| A012985_( | 1.6483 | 0.000317 | expressed protein |
| A023853_( | 2.4645 | 4.41E−05 | disease resistance protein (TIR class), |
| | | | putative |
| A017543_( | 1.6651 | 0.000419 | expressed protein/UVB-resistance protein- |
| | | | relate |
| A015908_( | 2.0363 | 7.39E−05 | ‘zinc finger (c3Hc4-type RING finger) |
| | | | protein |
| A010658_( | 1.9422 | 5.89 | delta 1-pyrroline-5-carboxylate synthetase B |
| | | | (P5 |
| A009505_( | 1.6179 | 5.89 | ‘ERD4 protein-related |
| A007555_( | 1.721 | 0.000259 | expressed protein |
| A022609_( | 1.7458 | 2.75 | ‘UDP = glycosyltransferase family |
| A011614_( | 1.6961 | 2.00E−05 | subtilisin-like serine protease |
| A012773_( | 2.7853 | 2.44E−06 | unknown |
| A008731_( | 1.7787 | 2.08E−06 | membrane channel protein-related |
| A021081_( | 2.0829 | 2.45E−05 | 29 kDa ribonucleoprotein, chloroplast (RNA- |
| | | | bin |
| A019523_( | 1.7551 | 7.14E−06 | subtilisin-like serine protease |
| A010673_( | 1.7384 | 5.83E−06 | expressed protein |
| A006796_( | 2.1806 | 9.75E−07 | glycosyl hydrolase family 19 (chitinase) |
| A017118_( | 1.7584 | 9.23E−06 | glycine-rich protein |
| A020107_( | 1.6579 | 2.18E−05 | ‘aluminium-induced protein-related |
| A001263_( | 1.8114 | 4.27E−15 | prolyl endopeptidase-related |
| A022887_( | 1.8946 | 1.96E−06 | RNA-binding protein, putative |
| A014704_( | 2.0674 | 3.15E−06 | glycine-rich RNA-binding protein 8 (GRP8) |
| | | | (CCI |
| A021522_( | 2.7332 | 8.85E−09 | expressed protein |
| A006636_( | 1.8637 | 1.82E−06 | photoropic response protein family |
| A010415_( | 1.7577 | 0.000435 | hypothetical protein |
| A015071_( | 2.5257 | 0.001612 | iron superoxide dismutase (FSD1) |
| A001387_( | 2.595 | 2.88E−05 | chloroplast nucleoid DNA binding protein- |
| | | | relate |
| A010894_( | 2.0208 | 3.68E−06 | tetrahydrofolate |
| | | | dehydrogenase/cyclohydrolase |
| A009216_( | 1.8682 | 0.000192 | expressed protein |
| A001231_( | 2.0434 | 1.47E−07 | monodehydroascorbate reductase, putative |
| A021731_( | 1.6457 | 0.005896 | hypothetical protein |
| A005991_( | 1.7641 | 1.39E−06 | 12S seed storage protein (CRA1) |
| A019461_( | 1.894 | 4.48E−07 | protein kinase, putative |
| A025241_( | 1.929 | 0.000284 | expressed protein |
| A015099_( | 1.714 | 0.015731 | glycoside hydrolase family 13 |
| A013821_( | 1.8172 | 7.80E−05 | galactosyltransferase family |
| A023088_( | 1.6601 | 0.000194 | gibberellin 20-oxidase, putative |
| A010858_( | 1.6227 | 0.000998 | hypothetical protein |
| A001251_( | 1.6799 | 0.000225 | hypothetical protein |

Table 7C

| | (AA)AATATCT | | | | | | |
|---|---|---|---|---|---|---|---|
| GeneID | 2000- | 1500 | 1500- | 1000 | 1000- | 500 | 500- |
| A020045_( | | | 1 | | | 1 | |
| A002062_( | | 1 | | | | | |
| A019512_( | | | | | | | |
| A001518_( | | | | | 1 | | |
| A017467_( | | | 1 | | | | |
| A018600_( | | | | | | | |
| A017042_( | | | | | | | 1 |
| A002820_( | | | | | | | |
| A023336_( | | | | | | | |
| A008602_( | | | | | | | |
| A021034_( | | | | | | 1 | |
| A020917_( | | | | | | 1 | |
| A003431_( | | | | | | | |
| A021216_( | 1 | | | | | | |
| A016420_( | | 1 | | | | | |
| A024454_( | | | | | | 1 | |
| A010449_( | | | | | | | |
| A001481_( | | | | | | | |
| A018891_( | | | | | 1 | | |
| A019105_( | | | | 2 | | | |
| A023455_( | 1 | 1 | | | | | |
| A019485_( | | | | | | | |
| A006869_( | | | | | | | |
| A005183_( | | | | | | 1 | |
| A000005_( | | | | | | | |
| A005372_( | | | | | | | |
| A012515_( | 1 | | | | | | |
| A008658_( | | | | 1 | | 3 | 1 |
| A018614_( | | | | | | | 1 |
| A001933_( | | | | | | | 1 |
| A001393_( | | | 1 | | 2 | 1 | |
| A005592_( | | | | | 2 | 1 | |
| A013749_( | | | | | 1 | 1 | |
| A019627_( | | | | 1 | 1 | | |
| A014046_( | | | | | 1 | | |
| A000325_( | | | | | 1 | 2 | |
| A021054_( | | | | | 1 | 1 | |
| A000993_( | | | | | 1 | | |
| A009934_( | | | | | 1 | | |
| A009127_( | | | | | 1 | | |
| A006288_( | | 2 | | | 1 | | |
| A020883_( | | | | | 1 | | |
| A009434_( | | 1 | | | 1 | | |
| A024703_( | | | | | 1 | | |
| A019449_( | | | 2 | | | 1 | |
| A020363_( | | | 1 | | | | |
| A020233_( | | | 1 | | | | |
| A017657_( | | 1 | 1 | | | | |
| A025734_( | | | 1 | | | | |
| A001363_( | | | 1 | | | | |
| A002862_( | | | 1 | 1 | | | |
| A003656_( | | 1 | 1 | | | | |
| A011016_( | 3 | 1 | | | | 1 | |
| A014515_( | 1 | 1 | | 1 | | | |
| A002709_( | 1 | | | | | | |
| A012985_( | 1 | | | | | | |
| A023853_( | 1 | | | 1 | | | |
| A017543_( | 1 | | | | | | |
| A015908_( | 1 | 1 | | | | | |
| A010658_( | 1 | | | | 1 | | |
| A009505_( | 1 | | | | | | |
| A007555_( | 1 | | | | 1 | | |
| A022609_( | 1 | | | | | | |
| A011614_( | 1 | | | 1 | 1 | | |
| A012773_( | | | | | | | |
| A008731_( | | | | | | | |
| A021081_( | | | | | | | |
| A019523_( | | | | | | | |
| A010673_( | | | | | 1 | | |
| A006796_( | | | | | 1 | | |
| A017118_( | | | 1 | | 2 | | |
| A020107_( | | | | | 1 | | |
| A001263_( | | | | | 1 | | |
| A022887_( | | 2 | | | 1 | | |
| A014704_( | | | 1 | 1 | | | |
| A021522_( | | | | 1 | | | |
| A006636_( | | | | 1 | | | |
| A010415_( | | | | 1 | | | |
| A015071_( | | | 1 | 1 | | | |
| A001387_( | | | | 1 | | | |
| A010894_( | | | | 1 | | | |
| A009216_( | | | | 1 | | | |
| A001231_( | | | | 1 | | | |
| A021731_( | | | 1 | | | | |
| A005991_( | | | 1 | | | | |
| A019461_( | | 1 | | | | | |
| A025241_( | | 1 | | | | | |
| A015099_( | | 1 | | | | | |
| A013821_( | | 1 | | | | | |
| A023088_( | | 1 | | | | | |
| A010858_( | | 1 | | | | | |
| A001251_( | | 1 | | | | | |

Table 7D

| GeneID | (AA)AATATCT | | | | | | | | |
|---|---|---|---|---|---|---|---|---|---|
| A020045_( | 400 | 400- | 300 | 300- | 200 | 200- | 100 | 100- | ATG |
| A002062_( | | | | 1 | | | 1 | 1 | |
| A019512_( | | | 1 | | | | | 1 | |
| A001518_( | | | | | | | | 1 | |
| A017467_( | | | | | | | | 1 | |
| A018600_( | | | | | | | | 1 | |
| A017042_( | | | | | | | | 1 | |
| A002820_( | | | | | | | | 1 | |
| A023336_( | | | | | | | | | |
| A008602_( | | | | | | | | | |
| A021034_( | | | | | | 2 | | | |
| A020917_( | | | | 1 | | 1 | | | 1 |
| A003431_( | | | | | | 1 | | | |
| A021216_( | | | | | | 1 | | | |
| A016420_( | | | | | | 1 | | | |
| A024454_( | | | | | | 1 | | | |
| A010449_( | | | | | | 1 | | | |
| A001481_( | | | | | | 1 | | | 1 |
| A018891_( | | | | | | 1 | | | |
| A019105_( | | | | | | 1 | | | |
| A023455_( | | | | | | 1 | | | |
| A019485_( | | | | | | 1 | | | |
| A006869_( | | | | 1 | | | | | |
| A005183_( | | | | 1 | | | | | |
| A000005_( | | | | 1 | | | | | |
| A005372_( | | 1 | | 1 | | | | | 1 |
| A012515_( | | 1 | | | | | | | |
| A008658_( | | | | | | | | | |
| A018614_( | | | | | | | | | |
| A001933_( | | | | | | | | | |
| A001393_( | | | | | | | | | |
| A005592_( | | | | | | | | | |
| A013749_( | | | | | | | | | |
| A019627_( | | | | | | | 1 | | |
| A014046_( | | | | | | | | | |
| A000325_( | | | | | 1 | | | | |
| A021054_( | | | | | | | | | |
| A000993_( | | | | | | | | | |
| A009934_( | 1 | | | | | | | | |
| A009127_( | | | | | 11 | | | | |
| A006288_( | | | | | | | 1 | | |
| A020883_( | | | | | | | | | |
| A009434_( | | | | | | | | | |
| A024703_( | | | | | | | | | |
| A019449_( | | | | | | | | | |
| A020363_( | 1 | | 1 | | | | | | |
| A020233_( | | | | | | | | | |
| A017657_( | | | | | | | | | 1 |
| A025734_( | | | | | | | | | |
| A001363_( | | | | | | | | | |
| A002862_( | | | | | | | | | |
| A003656_( | | | | | | | | | |
| A011016_( | | | | | | | | | |
| A014515_( | | | | | | | | | |
| A002709_( | | | | | | | | | |
| A012985_( | 2 | | | | | | | | |
| A023853_( | | | | | | | | | |
| A017543_( | | | | | | | | | |
| A015908_( | | | | | | | | | |
| A010658_( | | | | | | | | | |
| A009505_( | | | | | | | | | |
| A007555_( | | | | | | | | | |
| A022609_( | | | | | | | | | |
| A011614_( | | | | | | | | | 1 |
| A012773_( | | | | | | | | | |
| A008731_( | | | | | | | | | 1 |
| A021081_( | | | | | | | 1 | | 1 |
| A019523_( | | | | | | | 1 | | |
| A010673_( | 1 | | 1 | | | | 1 | | |
| A006796_( | | | | | 2 | | | | |
| A017118_( | | | | | 1 | | | | |
| A020107_( | | | | | | | | | |
| A001263_( | | | | | | | | | |
| A022887_( | | | | | | | | | |
| A014704_( | | | | | | | | | |
| A021522_( | | | | | | | | | |
| A006636_( | | | | | | | | | |
| A010415_( | | | | | | | | | |
| A015071_( | | | | | | | | | |
| A001387_( | | | | | | | | | |
| A010894_( | | | | | | | | | |
| A009216_( | | | | | | | | | |
| A001231_( | | | | | | | | | |
| A021731_( | | | | | | | | | |
| A005991_( | | | | | | | | | |
| A019461_( | | | | | | | | | |
| A025241_( | | | | | | | | | |
| A015099_( | | | | | | | | | |
| A013821_( | | | | | | | | | |
| A023088_( | | | | | | | | | |
| A010858_( | | | | | | | | | |
| A001251_( | | | | | | | | | |

[symbol]

indicates data missing or illegible when filed

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

The following references are all incorporated by reference to the extent they provide information available to one of ordinary skill in the art regarding the implementation of the technical teachings of the invention.
1. Wang, J. et al. Genomewide nonadditive gene regulation in Arabidopsis allotetraploids. Genetics 172, 507-517 (2006).
2. Lippman, Z. B. & Zamir, D. Heterosis: revisiting the magic. Trends Genet. 23, 60-66 (2007).
3. Birchler, J. A., Auger, D. L. & Riddle, N. C. In search of the molecular basis of heterosis. Plant Cell 15, 2236-2239. (2003).
4. Comai, L. et al. Phenotypic instability and rapid gene silencing in newly formed Arabidopsis allotetraploids. Plant Cell 12, 1551-1568 (2000).
5. Dodd, A. N. et al. Plant circadian clocks increase photosynthesis, growth, survival, and competitive advantage. Science 309, 630-633 (2005).
6. Wijnen, H. & Young, M. W. Interplay of circadian clocks and metabolic rhythms. Annu Rev Genet 40, 409-448 (2006).
7. Panda, S., Hogenesch, J. B. & Kay, S. A. Circadian rhythms from flies to human. Nature 417, 329-335 (2002).
8. Michael, T. P. et al. Enhanced fitness conferred by naturally occurring variation in the circadian clock. Science 302, 1049-1053 (2003).
9. Mizoguchi, T. et al. LHY and CCA1 are partially redundant genes required to maintain circadian rhythms in Arabidopsis. Dev Cell 2, 629-641 (2002).
10. Alabadi, D. et al. Reciprocal regulation between TOC1 and LHY/CCA1 within the Arabidopsis circadian clock. Science 293, 880-883 (2001).
11. Strayer, C. et al. Cloning of the Arabidopsis clock gene TOC1, an autoregulatory response regulator homolog. Science 289, 768-771 (2000).
12. Park, D. H. et al. Control of circadian rhythms and photoperiodic flowering by the Arabidopsis GIGANTEA gene. Science 285, 1579-1582 (1999).
13. Harmer, S. L. et al. Orchestrated transcription of key pathways in Arabidopsis by the circadian clock. Science 290, 2110-2113 (2000).
14. Leitch, A. R. & Leitch, I. J. Genomic plasticity and the diversity of polyploid plants. Science 320, 481-483 (2008).
15. Chen, Z. J. Genetic and epigenetic mechanisms for gene expression and phenotypic variation in plant polyploids. Annu Rev Plant Biol 58, 377-406 (2007).
16. Rieseberg, L. H. & Willis, J. H. Plant speciation. Science 317, 910-914 (2007).
17. Wang, J., Tian, L., Lee, H. S. & Chen, Z. J. Nonadditive Regulation of FRI and FLC Loci Mediates Flowering-Time Variation in Arabidopsis Allopolyploids. Genetics 173, 965-974 (2006).
18. Wang, Z. Y. & Tobin, E. M. Constitutive expression of the CIRCADIAN CLOCK ASSOCIATED 1 (CCA1) gene disrupts circadian rhythms and suppresses its own expression. Cell 93, 1207-1217 (1998).
19. Alabadi, D., Yanovsky, M. J., Mas, P., Harmer, S. L. & Kay, S. A. Critical role for CCA1 and LHY in maintaining circadian rhythmicity in Arabidopsis. Curr Biol 12, 757-761 (2002).
20. McClung, C. R. Plant circadian rhythms. Plant Cell 18, 792-803 (2006).
21. Jenuwein, T. & Allis, C. D. Translating the histone code. Science 293, 1074-1080 (2001).
22. Doyle, M. R. et al. The ELF4 gene controls circadian rhythms and flowering time in Arabidopsis thaliana. Nature 419, 74-77 (2002).
Reinbothe, S., Reinbothe, C., Lebedev, N. & Apel, K. PORA and PORB, Two Light-Dependent Protochlorophyllide-Reducing Enzymes of Angiosperm Chlorophyll Biosynthesis. Plant Cell 8, 763-769 (1996).
24. Sperling, U., van Cleve, B., Frick, G., Apel, K. & Armstrong, G. A. Overexpression of light-dependent PORA or PORB in plants depleted of endogenous POR by far-red light enhances seedling survival in white light and protects against photooxidative damage. Plant J 12, 649-658 (1997).
25. Lloyd, J. R., Kossmann, J. & Ritte, G. Leaf starch degradation comes out of the shadows. Trends Plant Sci 10, 130-137 (2005).
26. Smith, A. M., Zeeman, S. C. & Smith, S. M. Starch degradation Annu Rev Plant Biol 56, 73-98 (2005).
27. Smith, S. M. et al. Diurnal changes in the transcriptome encoding enzymes of starch metabolism provide evidence for both transcriptional and posttranscriptional regulation of starch metabolism in Arabidopsis leaves. Plant Physiol 136, 2687-2699 (2004).
28. Eimert, K., Wang, S. M., Lue, W. I. & Chen, J. Monogenic Recessive Mutations Causing Both Late Floral Initiation and Excess Starch Accumulation in Arabidopsis. Plant Cell 7, 1703-1712 (1995).
29. Hall, A. et al. The TIME FOR COFFEE gene maintains the amplitude and timing of Arabidopsis circadian clocks. Plant Cell 15, 2719-2729 (2003).
30. Perales, M. & Mas, P. A functional link between rhythmic changes in chromatin structure and the Arabidopsis biological clock. Plant Cell 19, 2111-2123 (2007).
31. Madlung, A. et al. Remodeling of DNA methylation and phenotypic and transcriptional changes in synthetic Arabidopsis allotetraploids. Plant Physiol 129, 733-746 (2002).
32. Wang, J. et al. Stochastic and epigenetic changes of gene expression in Arabidopsis polyploids. Genetics 167, 1961-1973 (2004).
33. Clough, S. J. & Bent, A. F. Floral dip: a simplified method for Agrobacterium-mediated transformation of Arabidopsis thaliana. Plant J 16, 735-743 (1998).
34. Lee, H. S. et al. Sensitivity of 70-mer oligonucleotides and cDNAs for microarray analysis of gene expression in Arabidopsis and its related species. Plant Biotechnology Journal 2, 45-57 (2004).
35. Mochizuki, N., Brusslan, J. A., Larkin, R., Nagatani, A. & Chory, J. Arabidopsis genomes uncoupled 5 (GUN5) mutant reveals the involvement of Mg-chelatase H subunit in plastid-to-nucleus signal transduction. Proc Natl Acad Sci USA 98, 2053-2058 (2001).
36. Yu, T. S. et al. The Arabidopsis sexl mutant is defective in the R1 protein, a general regulator of starch degradation in plants, and not in the chloroplast hexose transporter. Plant Cell 13, 1907-1918 (2001).
37. Smith, A. M. & Zeeman, S. C. Quantification of starch in plant tissues. Nat Protoc 1, 1342-1345 (2006).
38. Focks, N. & Benning, C. wrinkled1: A novel, low-seed-oil mutant of Arabidopsis with a deficiency in the seed-specific regulation of carbohydrate metabolism. Plant Physiol 118, 91-101 (1998).
39. Harmer, S. L. & Kay, S. A. Positive and negative factors confer phase-specific circadian regulation of transcription in Arabidopsis. Plant Cell 17, 1926-1940 (2005).
40. Arabidopsis Genome Initiative. Analysis of the genome sequence of the flowering plant Arabidopsis thaliana. Nature 408, 796-815 (2000).
41. Bastow, R. et al. Vernalization requires epigenetic silencing of FLC by histone methylation. Nature 427, 164-167 (2004).
42. Tian, L. et al. Reversible histone acetylation and deacetylation mediate genome-wide, promoter-dependent and locus-specific changes in gene expression during plant development. Genetics 169, 337-345 (2005).
43. Pruneda-Paz, J. L., et al. A functional genomics approach reveals CHE as a component of the Arabidopsis circadian clock. Science 323, 1481-1485 (2009).
44. Murakami M, Tago Y, Yamashino T, Mizuno T: Characterization of the rice circadian clock-associated pseudo-response regulators in Arabidopsis thaliana. Biosci Biotechnol Biochem 71:1107-1110 (2007).
45. Murakami M, Tago Y, Yamashino T, Mizuno T: Comparative overviews of clock-associated genes of Arabidopsis thaliana and Oryza sativa. Plant Cell Physiol, 48:110-121 (2007).
46. Swanson-Wagner, R. A. et al. All possible modes of gene action are observed in a global comparison of gene expression in a maize F1 hybrid and its inbred parents. Proc Natl Acad Sci USA 103, 6805-6810 (2006).
47. Fukayama, H. et al. Characterization and functional analysis of phosphoenolpyruvate carboxylase kinase genes in rice. Plant J 47, 258-268 (2006).
48. Shenton, M. et al. Distinct patterns of control and expression amongst members of the PEP carboxylase kinase gene family in C4 plants. Plant J 48, 45-53 (2006),
49. Alonso-Blanco, C., et al. Natural allelic variation at seed size loci in relation to other life history traits of Arabidopsis thaliana. Proc Natl Acad Sci USA 96, 4710-4717 (1999).
50. el-Assal, S. E., et al. Pleiotropic effects of the Arabidopsis cryptochrome 2 allelic variation underlie fruit trait-related QTL. Plant Biol (Stuttg) 6, 370-374 (2004).
51. van der Horst, G. T. et al. Mammalian Cry1 and Cry2 are essential for maintenance of circadian rhythms. Nature 398, 627-630 (1999).
52. Griffin, E. A., Jr., Staknis, D. & Weitz, C. J. Light-independent role of CRY1 and CRY2 in the mammalian circadian clock. Science 286, 768-771 (1999).
53. Kume, K. et al. mCRY1 and mCRY2 are essential components of the negative limb of the circadian clock feedback loop. Cell 98, 193-205 (1999).

1-33. (canceled)

34. A method for promoting growth vigor in a plant comprising:
providing a plant comprising the CIRCADIAN CLOCK ASSOCIATED 1 (CCA1) circadian clock gene; and
modifying expression of the circadian clock gene by inhibiting expression of CCA1 or LATE ELONGATED HYPOCOTYL (LHY) by overexpressing at least one of CCA1 HIKING EXPEDITION(CHE), E74-LIKE FACTOR 3 (ELF3), LUCIFERASE (LUX), PHYTOCHROME (PHY), or TIME FOR COFFEE (TIC), administering a transcription inhibitor, or administering a translation inhibitor.

35. The method of claim 34, wherein the plant is a hybrid or a polyploid.

36. The method of claim 34, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.

37. A method for promoting growth vigor in a plant comprising:
providing a plant comprising the CIRCADIAN CLOCK ASSOCIATED 1 (CCA1) circadian clock gene; and
modifying the activity of a protein encoded by the circadian clock gene by administering a CCA1 or LATE ELONGATED HYPOCOTYL (LHY) inhibitor or administering a chromatin reagent or enhancing expression of CCA1 HIKING EXPEDITION (CHE), so as to modify a flowering time of the plant, modify a starch, sugar, chlorophyll, metabolite, or nutrient content of the plant, or increase the biomass of the plant.

38. The method of claim 37, wherein enhancing the expression of CHE comprises administering a CHE enhancer or increasing a promoter element of CHE.

39. The method of claim 37, wherein the plant is a hybrid or a polyploid.

40. The method of claim 37, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.

41. A method for promoting growth vigor in a plant comprising:
providing a plant comprising the LATE ELONGATED HYPOCOTYL (LHY) circadian clock gene; and
modifying expression of the circadian clock gene by inhibiting expression of CIRCADIAN CLOCK ASSOCIATED 1 (CCA1) or LHY by overexpressing at least one of CCA1 HIKING EXPEDITION(CHE), E74-LIKE FACTOR 3 (ELF3), LUCIFERASE (LUX), PHYTOCHROME (PHY), or TIME FOR COFFEE (TIC), administering a transcription inhibitor, or administering a translation inhibitor.

42. The method of claim 41, wherein the plant is a hybrid or a polyploid.

43. The method of claim 41, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.

44. A method for promoting growth vigor in a plant comprising:
providing a plant comprising the LATE ELONGATED HYPOCOTYL (LHY) circadian clock gene; and
modifying the activity of a protein encoded by the circadian clock gene by administering a CIRCADIAN CLOCK ASSOCIATED 1 (CCA1) or LHY inhibitor or administering a chromatin reagent or enhancing expression of CCA1 HIKING EXPEDITION(CHE), so as to modify a flowering time of the plant, modify a starch, sugar, chlorophyll, metabolite, or nutrient content of the plant, or increase the biomass of the plant.

45. The method of claim 44, wherein enhancing the expression of CHE comprises administering a CHE enhancer or increasing a promoter element of CHE.

46. The method of claim 44, wherein the plant is a hybrid or a polyploid.

47. The method of claim 44, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.

48. A method for promoting growth vigor in a plant comprising:
providing a plant comprising the CCA1 HIKING EXPEDITION(CHE) circadian clock gene; and
modifying expression of the circadian clock gene by inhibiting expression of CIRCADIAN CLOCK ASSOCIATED 1 (CCA1) or LATE ELONGATED HYPOCOTYL (LHY) by overexpressing at least one of CHE, E74-LIKE FACTOR 3 (ELF3), LUCIFERASE (LUX), PHYTOCHROME (PHY), or TIME FOR COFFEE (TIC), administering a transcription inhibitor, or administering a translation inhibitor.

49. The method of claim 48, wherein the plant is a hybrid or a polyploid.

50. The method of claim 48, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.

51. A method for promoting growth vigor in a plant comprising:
providing a plant comprising the CCA1 HIKING EXPEDITION(CHE) circadian clock gene; and
modifying the activity of a protein encoded by the circadian clock gene by administering a CIRCADIAN CLOCK ASSOCIATED 1 (CCA1) or LATE ELONGATED HYPOCOTYL (LHY) inhibitor or administering a chromatin reagent or enhancing expression of CHE, so as to modify a flowering time of the plant, modify a starch, sugar, chlorophyll, metabolite, or nutrient content of the plant; or increase the biomass of the plant.

52. The method of claim 51, wherein enhancing the expression of CHE comprises administering a CHE enhancer or increasing a promoter element of CHE.

53. The method of claim 51, wherein the plant is a hybrid or a polyploid.

54. The method of claim 51, wherein the plant cell is corn, wheat, rice, sugarcane, sorghum, millet, rye, cotton, soybean, tobacco, oilseed rape, spinach, a grape, sunflower, a peanut, mustard, a vegetable, a fruit, a pepper, a tomato, a cucumber, a squash, a potato, a cabbage, an onion, a rose, a petunia, a strawberry, a peach, an apple, an orange, a banana, coca, cassaya, switchgrass, elephant grass, Sudan grass, Chinese tallow, clover, Jatropha curcas, algae, a tree, tea tree, a bamboo tree, a poplar tree, a willow tree, a palm tree, a pine tree, ginseng, ginger, ginko, motherwort, berberis, or Coptis.


SEQUENCE LISTING (99 sequences)

SEQ ID NO: 1 - DNA, 33 bp, Artificial Sequence
  DNA Primer
gggaattccgtgtcttacggtggatgaagttga

SEQ ID NO: 2 - DNA, 42 bp, Artificial Sequence
  DNA Primer
ggggatccgttttgtcaatcaatggtcaaattatgagacgcg

SEQ ID NO: 3 - DNA, 34 bp, Artificial Sequence
  DNA Primer
gcggccggatccatggagacaaattcgtctggag

SEQ ID NO: 4 - DNA, 33 bp, Artificial Sequence
  DNA Primer
ggccgctctagatcatgtggaagcttgagtttc

SEQ ID NO: 5 - DNA, 56 bp, Artificial Sequence
  DNA Primer
ggggacaagtttgtacaaaaaagcaggcttacgtgtcttacggtggatgaagttga

SEQ ID NO: 6 - DNA, 54 bp, Artificial Sequence
  DNA Primer
ggggaccactttgtacaagaaagctgggtctgtggaagcttgagtttccaaccg

SEQ ID NO: 7 - DNA, 33 bp, Artificial Sequence; identical to SEQ ID NO: 1
  DNA Primer

SEQ ID NO: 8 - DNA, 46 bp, Artificial Sequence
  DNA Primer
gcggccccatgggttttgtcaatcaatggtcaaattatgagacgcg

SEQ ID NO: 9 - DNA, 45 bp, Artificial Sequence
  DNA Primer
gcggcctctagaggcgcgcctctggaaaacggtaatgagcaagga

SEQ ID NO: 10 - DNA, 44 bp, Artificial Sequence
  DNA Primer
ggccgccctaggtaaatttacaccactagaatcgggaggccaaa

SEQ ID NO: 11 - DNA, 9 bp, Artificial Sequence
  Evening Element
aaaatatct

SEQ ID NO: 12 - DNA, 8 bp, Artificial Sequence
  CCA1 binding site
aaaaatct

SEQ ID NO: 13 - DNA, 55 bp, Artificial Sequence
  DNA Primer
ggggacaagtttgtacaaaaaagcaggctccctcgagatggagacaaattcgtct

SEQ ID NO: 14 - DNA, 58 bp, Artificial Sequence
  DNA Primer
ggggaccactttgtacaagaaagctgggtcccctaggtcatgtggaagcttgagtttc

SEQ ID NO: 15 - DNA, 22 bp, Artificial Sequence
  DNA Primer
cctcgtcagacacagacttcca

SEQ ID NO: 16 - DNA, 23 bp, Artificial Sequence
  DNA Primer
ccgcagtagaatcagctccaata

SEQ ID NO: 17 - DNA, 23 bp, Artificial Sequence
  DNA Primer
gggacaaagactgctgttcagat

SEQ ID NO: 18 - DNA, 24 bp, Artificial Sequence
  DNA Primer
tttgtgaagaacttttgtgcatga

SEQ ID NO: 19 - DNA, 20 bp, Artificial Sequence
  DNA Primer
gttgatggatcgggtttctc

SEQ ID NO: 20 - DNA, 20 bp, Artificial Sequence
  DNA Primer
tcatgaccccatgcatacag

SEQ ID NO: 21 - DNA, 23 bp, Artificial Sequence
  DNA Primer
tcgagcaacttcatcatcacaaa

SEQ ID NO: 22 - DNA, 24 bp, Artificial Sequence
  DNA Primer
gctaatggagctggtgtcatactg

SEQ ID NO: 23 - DNA, 22 bp, Artificial Sequence
  DNA Primer
gtctgtgacaatggaactggaa

SEQ ID NO: 24 - DNA, 22 bp, Artificial Sequence
  DNA Primer
ctttctgacccataccaaccat

SEQ ID NO: 25 - DNA, 22 bp, Artificial Sequence
  DNA Primer
ggatcggttatattggagctga

SEQ ID NO: 26 - DNA, 22 bp, Artificial Sequence
  DNA Primer
aacacctgagagtgatgcaaag

SEQ ID NO: 27 - DNA, 24 bp, Artificial Sequence
  DNA Primer
ttgatcttgcagagctgtgttttg

SEQ ID NO: 28 - DNA, 22 bp, Artificial Sequence
  DNA Primer
cttggtgggcttctcatggact

SEQ ID NO: 29 - DNA, 20 bp, Artificial Sequence
  DNA Primer
gcgccagctatccacattcc

SEQ ID NO: 30 - DNA, 20 bp, Artificial Sequence
  DNA Primer
cttggttcatgaccccatgc

SEQ ID NO: 31 - DNA, 22 bp, Artificial Sequence
  DNA Primer
atcactgatccatggagacaaa

SEQ ID NO: 32 - DNA, 21 bp, Artificial Sequence
  DNA Primer
gagaaatccttcgcattttga

SEQ ID NO: 33 - DNA, 21 bp, Artificial Sequence
  DNA Primer
ctcatgaagattctcactgag

SEQ ID NO: 34 - DNA, 22 bp, Artificial Sequence
  DNA Primer
acaacagatagttcaattccca

SEQ ID NO: 35 - DNA, 22 bp, Artificial Sequence
  DNA Primer
ggagaaatctcagccactataa

SEQ ID NO: 36 - DNA, 20 bp, Artificial Sequence
  DNA Primer
aactcgtggcctagaataca

SEQ ID NO: 37 - DNA, 22 bp, Artificial Sequence
  DNA Primer
gctggaacagcaccaagggtat

SEQ ID NO: 38 - DNA, 22 bp, Artificial Sequence
  DNA Primer
ctggcaccgtacccacttgttt

SEQ ID NO: 39 - DNA, 21 bp, Artificial Sequence
  DNA Primer
aaacgaaacgaagccgaatcc

SEQ ID NO: 40 - DNA, 22 bp, Artificial Sequence
  DNA Primer
ggtcaaattatgagacgcgaaa

SEQ ID NO: 41 - DNA, 18 bp, Artificial Sequence
  DNA Primer
atggtaatggcgcataaa

SEQ ID NO: 42 - DNA, 17 bp, Artificial Sequence
  DNA Primer
caaatgattcgggaaaa

SEQ ID NO: 43 - DNA, 25 bp, Artificial Sequence
  DNA Primer
cgtttcgctttccttagtgttagct

SEQ ID NO: 44 - DNA, 27 bp, Artificial Sequence
  DNA Primer
agcgaacggatctagagactcaccttg

SEQ ID NO: 45 - DNA, 22 bp, Artificial Sequence
  DNA Primer
aaaccatttgggccactttctt

SEQ ID NO: 46 - DNA, 22 bp, Artificial Sequence
  DNA Primer
caagtcttttcccagcctctga

SEQ ID NO: 47 - DNA, 22 bp, Artificial Sequence
  DNA Primer
accaaatcaaatccgaacatgg

SEQ ID NO: 48 - DNA, 22 bp, Artificial Sequence
  DNA Primer
ggctctttagctgtcgggaaat

SEQ ID NO: 49 - DNA, 21 bp, Artificial Sequence
  DNA Primer
ttcctcctttgctttggcgta

SEQ ID NO: 50 - DNA, 20 bp, Artificial Sequence
  DNA Primer
tccagtggacgggaggaaaa

SEQ ID NO: 51 - DNA, 22 bp, Artificial Sequence
  DNA Primer
gccattgttgcagctctccttt

SEQ ID NO: 52 - DNA, 22 bp, Artificial Sequence
  DNA Primer
ttccaactcacaaacccatcca

SEQ ID NO: 53 - DNA, 22 bp, Artificial Sequence
  DNA Primer
cggtggaggtaaccacagaaca

SEQ ID NO: 54 - DNA, 20 bp, Artificial Sequence
  DNA Primer
aaactggctgcggaggcata

SEQ ID NO: 55 - DNA, 22 bp, Artificial Sequence
  DNA Primer
gccatgtttggcatgtgttctt

SEQ ID NO: 56 - DNA, 22 bp, Artificial Sequence
  DNA Primer
aaaacctcgcacatgcatttca

SEQ ID NO: 57 - DNA, 22 bp, Artificial Sequence
  DNA Primer
tcccaagactcacaaacccaca

SEQ ID NO: 58 - DNA, 22 bp, Artificial Sequence
  DNA Primer
tgaagcatgccaaacatcacct

SEQ ID NO: 59 - DNA, 24 bp, Artificial Sequence
  DNA Primer
gcatctacaatgacggagacgaaa

SEQ ID NO: 60 - DNA, 22 bp, Artificial Sequence
  DNA Primer
gccattctccaggaccatcaac

SEQ ID NO: 61 - DNA, 23 bp, Artificial Sequence
  DNA Primer
cgccgctgattttaatcttcgat

SEQ ID NO: 62 - DNA, 22 bp, Artificial Sequence
  DNA Primer
tcgattccatcttcgctgaatg

SEQ ID NO: 63 - DNA, 22 bp, Artificial Sequence
  DNA Primer
ccaaaaccctgcaaatcctctg

SEQ ID NO: 64 - DNA, 22 bp, Artificial Sequence
  DNA Primer
aaaggcagtggcagaaagttcg

SEQ ID NO: 65 - DNA, 22 bp, Artificial Sequence
  DNA Primer
gcagcagcagaatatgcaagga

SEQ ID NO: 66 - DNA, 20 bp, Artificial Sequence
  DNA Primer
cgccaccaggcatagttgtc

SEQ ID NO: 67 - DNA, 22 bp, Artificial Sequence
  DNA Primer
attatccggcttggggttatgg

SEQ ID NO: 68 - DNA, 22 bp, Artificial Sequence
  DNA Primer
tcggaaggagcttttgttgacc

SEQ ID NO: 69 - DNA, 22 bp, Artificial Sequence
  DNA Primer
agctgcctcttgtattcgtgga

SEQ ID NO: 70 - DNA, 22 bp, Artificial Sequence
  DNA Primer
tgaatggcgttgctcagttaca

SEQ ID NO: 71 - DNA, 22 bp, Artificial Sequence
  DNA Primer
ggtgaatcgaagaaaggcgatg

SEQ ID NO: 72 - DNA, 22 bp, Artificial Sequence
  DNA Primer
catggttccttcttccccactg

SEQ ID NO: 73 - DNA, 22 bp, Artificial Sequence
  DNA Primer
ttttggggcagaggacctgata

SEQ ID NO: 74 - DNA, 23 bp, Artificial Sequence
  DNA Primer
gagcctctgggaaatcctcatgt

SEQ ID NO: 75 - DNA, 22 bp, Artificial Sequence
  DNA Primer
gccttcaaatcgttcacggaag

SEQ ID NO: 76 - DNA, 22 bp, Artificial Sequence
  DNA Primer
aaaggagctggtgtggaaggtg

SEQ ID NO: 77 - DNA, 22 bp, Artificial Sequence
  DNA Primer
ggtgttcatggaatcgcagttg

SEQ ID NO: 78 - DNA, 22 bp, Artificial Sequence
  DNA Primer
gccaccgaacaaaggaagttga

SEQ ID NO: 79 - DNA, 22 bp, Artificial Sequence
  DNA Primer
tttgcgtttcagagacggacct

SEQ ID NO: 80 - DNA, 22 bp, Artificial Sequence
  DNA Primer
agcaccaagacaagcaacacca

SEQ ID NO: 81 - DNA, 22 bp, Artificial Sequence
  DNA Primer
tcccacagttgccacacagttt

SEQ ID NO: 82 - DNA, 22 bp, Artificial Sequence
  DNA Primer
ggtggagcaaacattccgtttc

SEQ ID NO: 83 - DNA, 21 bp, Artificial Sequence
  DNA Primer
ctcatgaagattctcactgag

SEQ ID NO: 84 - DNA, 22 bp, Artificial Sequence
  DNA Primer
acaacagatagttcaattccca

SEQ ID NO: 85 - DNA, 25 bp, Artificial Sequence
  DNA Primer
cacaaacaaaaaaatctcttatcac

SEQ ID NO: 86 - DNA, 25 bp, Artificial Sequence
  DNA Primer
gtgataagagatttttttgtttgtg

SEQ ID NO: 87 - DNA, 25 bp, Artificial Sequence
  DNA Primer
agagcaacaaaatatctcgactgtt

SEQ ID NO: 88 - DNA, 25 bp, Artificial Sequence
  DNA Primer
aacagtcgagatattttgttgctct

SEQ ID NO: 89 - DNA, 25 bp, Artificial Sequence
  DNA Primer
tatacattaaaatatctactgacag

SEQ ID NO: 90 - DNA, 25 bp, Artificial Sequence
  DNA Primer
ctgtcagtagatattttaatgtata

SEQ ID NO: 91 - DNA, 25 bp, Artificial Sequence
  DNA Primer
attaaaataaaatatctaaggagaa

SEQ ID NO: 92 - DNA, 25 bp, Artificial Sequence
  DNA Primer
ttctccttagatattttattttaat

SEQ ID NO: 93 - DNA, 25 bp, Artificial Sequence
  DNA Primer
acacaaaaaaaatatctaatcacag

SEQ ID NO: 94 - DNA, 25 bp, Artificial Sequence
  DNA Primer
ctgtgattagatattttttttgtgt

SEQ ID NO: 95 - DNA, 24 bp, Artificial Sequence
  DNA Primer
ttggtttctgatggtttggtctga

SEQ ID NO: 96 - DNA, 24 bp, Artificial Sequence
  DNA Primer
cgcttgacccatagctacaccttt

SEQ ID NO: 97 - DNA, 24 bp, Artificial Sequence
  DNA Primer
ttggtttctgatggtttggtctga

SEQ ID NO: 98 - DNA, 28 bp, Artificial Sequence
  DNA Primer
gaacccgtttgggtgagcttaaaagtgg

SEQ ID NO: 99 - DNA, 27 bp, Artificial Sequence
  DNA Primer
aagggatgacgcacaatcccactatcc